(19) United States
(12) Patent Application Publication
(10) Pub. No.: US 2026/0266804 A1
(43) Pub. Date: Sep. 10, 2026

(54) MODEL FOR ANALYZING INFLAMMATION AND USES THEREOF

(71) Applicant: Technion Research & Development Foundation Limited, Haifa (IL)

(72) Inventors: Esther MEYRON-HOLTZ, Haifa (IL); Lulu FAHOUM, Haifa (IL)

(73) Assignee: Technion Research & Development Foundation Limited, Haifa (IL)

(21) Appl. No.: 18/993,630

(22) PCT Filed: Jul. 11, 2023
(86) PCT No.: PCT/IL2023/050723

§ 371 Date: Jan. 13, 2025

Related U.S. Application Data
(60) Provisional application No. 63/388,664, filed on Jul. 13, 2022.

(51) Int. Cl. G01N 33/50; G01N 33/68

(52) U.S. Cl. CPC G01N 33/5047; G01N 33/5023; G01N 33/5058; G01N 33/5067; G01N 33/6893; G01N 2333/52; G01N 2333/70582; G01N 2333/988

(57) ABSTRACT

An in vitro culture model for inflammation is provided. The method comprises tissue cells and immune cells, wherein the tissue cells and immune cells are of different species and are in direct contact. Also provided is an in vitro method of assaying inflammation in culture using the model.

### RELATED APPLICATION/S

This application claims the benefit of priority of U.S. Provisional Patent Application No. 63/388,664 filed on Jul. 13, 2022, the contents of which are incorporated herein by reference in their entirety.

### SEQUENCE LISTING STATEMENT

The XML file, entitled 96652 Sequence Listing.xml, created on 11 Jul. 2023, comprising 50,593 bytes, submitted concurrently with the filing of this application is incorporated herein by reference.

### FIELD AND BACKGROUND OF THE INVENTION

The present invention, in some embodiments thereof, relates to a model for analyzing inflammation and uses thereof.

Inflammatory diseases, such as inflammatory bowel diseases (IBD), are complex disorders that are treated mostly by suppressing activated inflammatory pathways of the immune system. Recently, mitochondrial defects and impaired energy homeostasis were identified as possible drivers of the inflammation, suggesting an involvement of iron homeostasis in the pathophysiology of IBD and other inflammatory conditions. In the inflamed tissue of IBD-patients, iron accumulation was demonstrated, but neither a mechanism for the accumulation nor its function in the course of inflammation were known. Although the cellular and molecular events initiating and perpetuating the inflammation are not fully understood, it is clear, that intestinal homeostasis is broadly disrupted, causing a pathological inflammation that leads to tissue injury. Iron was previously found to accumulate in the inflamed tissues of IBD (Crohn's Disease (CD) and Colitis) patients compared to non-inflamed areas of the same patients and of healthy individuals,² but neither the role nor the mechanism of this iron accumulation are known. Iron is an essential nutrient, however unbalanced iron levels lead to inflammation and tissue damage.³⁻⁵ Iron homeostasis is tightly regulated, at the systemic level mainly by hepcidin and at the cellular level, mainly by the iron regulatory proteins (IRPs)1 and 2.⁶ When cellular iron is low, IRPs bind with high affinity to RNA motifs known as iron-responsive elements (IREs), which are found in numerous transcripts, mostly related to iron homeostasis. IRP2 is degraded by high iron, oxygen and ROS/RNS levels.⁷ IRP1 is a bifunctional protein changing conformation from a cytosolic iron sulfur-cluster containing aconitase (cAco) at high cellular iron to an RNA (IRE)-binding apoprotein, when cellular iron is low. The iron mediated conformational change depends strongly on the presence of oxygen or reactive oxygen or nitrogen species (ROS/RNS).⁸ At physiologic redox state and oxygen conditions, IRP1 is mainly in its non-IRE-binding (cAco) state, and does not change to its RNA-binding form, even when iron is low.^{9,10} As IRP2 is highly regulated by iron at physiologic conditions, it is regarded the dominant iron regulator.¹¹

Many of the published setups that all included Caco2 cells as a model for an absorptive enterocyte, in various combinations, and mimicked well-established inflammatory parameters upon induction of inflammation with appropriate stimuli, did not mimic the inflammatory iron pattern. Clearly a model for inflammation and specifically gastrointestinal inflammation which addresses iron homeostasis and its role in inflammation would be of great value in assessing inflammation and its modulation.

### RELATED BACKGROUND ART

Antunes, F., Andrade, F., Araujo, F., Ferreira, D. & Sarmento, B. Establishment of a triple co-culture in vitro cell models to study intestinal absorption of peptide drugs. European journal of pharmaceutics and biopharmaceutics: official journal of Arbeitsgemeinschaft fur Pharmazeutische Verfahrenstechnik e.V 83, 427-435, doi:10.1016/j.ejpb.2012.10.003 (2013).
Tanoue, T., Nishitani, Y., Kanazawa, K., Hashimoto, T. & Mizuno, M. In vitro model to estimate gut inflammation using co-cultured Caco-2 and RAW264.7 cells. Biochem Biophys Res Commun 374, 565-569 (2008)

### SUMMARY OF THE INVENTION

According to an aspect of some embodiments of the present invention there is provided an in vitro culture model for inflammation comprising tissue cells and immune cells, wherein the tissue cells and immune cells are of different species and are in direct contact.

According to an aspect of some embodiments of the present invention there is provided an in vitro method of assaying inflammation in culture, the method comprising:
(a) contacting the model as described herein with a test agent; and
(b) analysing an inflammation parameter in the tissue cells and/or immune cells.

According to some embodiments of the invention, the inflammation parameter is associated with iron homeostasis.

According to some embodiments of the invention, the inflammation parameter associated with iron homeostasis is iron amount or an indicator thereof in the tissue cells and/or immune cells.

According to some embodiments of the invention, an indication of inflammation by the iron amount is when iron amount is increased in immune cells with respect to non-inflamed immune cells of the same type and/or an iron amount is altered in the tissue cells with respect to non-inflamed tissue cells of the same type.

According to some embodiments of the invention, the inflammation parameter associated with iron homeostasis is an iron-regulated gene.

According to some embodiments of the invention, the iron-regulated gene is selected from the group consisting of ferritin, Transferrin Receptor (TfR)1, NCOA4, IRP1 and IRP2.

According to some embodiments of the invention, the analyzing is following at least 4 hours from the contacting.

According to some embodiments of the invention, the analyzing expression of the iron-regulated gene is at the protein level.

According to some embodiments of the invention, the analyzing expression of the iron-regulated gene is at the mRNA level.

According to some embodiments of the invention, the iron-regulated gene is ferritin and the analysing is by analysing ferritin-iron complex in the immune cells and wherein inflammation is indicated by an increase in the ferritin-iron complex in the immune cells, wherein the increase is with respect to control non-inflamed cells of the same type.

According to some embodiments of the invention, the iron-regulated protein is TfR1, wherein inflammation is indicated by a decrease in the TfR1 expression in the immune cells, wherein the decrease is with respect to control non-inflamed cells of the same type.

According to some embodiments of the invention, the iron-regulated protein is IRP2, wherein inflammation is indicated by a decrease in the IRP2 expression in the immune cells, wherein decrease is with respect to control non-inflamed cells of the same type.

According to some embodiments of the invention, the iron-related protein is ferritin and analyzing is by analyzing H-subunit (FTH1) and L-subunit (FTL) thereof, wherein inflammation is indicated wherein FTH1:FTL protein ratio above 1 preferably above 2 in the immune cells.

According to some embodiments of the invention, the inflammation parameter associated with iron homeostasis is a decrease in cellular or mitochondrial aconitase activity of IRP1 in the immune cells and the tissue cells, wherein the decrease is with respect to control non-inflamed cells of the same type.

According to some embodiments of the invention, the inflammation parameter associated with iron homeostasis is a protein involved in Fe—S cluster assembly and wherein inflammation is indicated when a decrease in the immune cells is indicated, wherein a decrease is with respect to control non-inflamed cells of the same type.

According to some embodiments of the invention, gene is Nfs1 or IscU.

According to some embodiments of the invention, the tissue cells are GI cells.

According to some embodiments of the invention, the tissue cells are epithelial cells or fibroblasts.

According to some embodiments of the invention, the tissue cells are epithelial cells.

According to some embodiments of the invention, the epithelial cells have a decreased iron amount with respect to non-inflamed epithelial cells of the same type, and optionally wherein the tissue I a GI tissue, an eye tissue and a brain tissue.

According to some embodiments of the invention, the tissue cells are selected from the group consisting of hepatocytes, neurons, cartilage, osteoblasts and fibroblasts.

According to some embodiments of the invention, the tissue cells have a decreased iron amount with respect to non-inflamed epithelial cells of the same type, and optionally wherein the tissue comprises an epithelial barrier.

According to some embodiments of the invention, said tissue is selected from the group consisting of a testical tissue, an eye tissue and a brain tissue.

According to some embodiments of the invention, said inflammation parameter is a pro-inflammatory cytokine and/or an anti-inflammatory cytokine, wherein an increase in said pro-inflammatory cytokine and/or a decrease in anti-inflammatory cytokine in said immune cells and/or tissue cells is indicative of inflammation, wherein said increase or decrease is with respect to control non-inflamed cells of the same type.

According to some embodiments of the invention, said inflammation parameter is a signalling pathway associated with inflammation.

According to some embodiments of the invention, said signalling is a MAP-kinase pathway, wherein an increase in signalling via said MAP-kinase pathway n said immune cells and/or tissue cells is indicative of inflammation, wherein said increase is with respect to control non-inflamed cells of the same type.

According to an aspect of some embodiments of the present invention there is provided the model or method as described herein for use in drug screening.

According to an aspect of some embodiments of the present invention there is provided the model or method as described herein for use in qualifying consumables in food industry.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show that inflammation causes a specific pattern of iron distribution in the terminal ileum (TI). A. Ferritin is decreased in the epithelial layer of the inflamed TI. Healthy and inflamed intestinal sections of Crohn's disease patients (n=8), and of 12-14 weeks old C57BL/6 (WT, control) and TNF^{ΔARE/+} on C57BL/6 background mice (inflamed, TNF^{ΔARE/+}) (n=3), were stained for ferritin (green). Representative images show that the epithelial cells (IEC) (arrows) have decreased ferritin levels in both human and mouse sections. Further, in the TNF^{ΔARE/+} mice, the lamina propria cells (LP) show an increase in ferritin levels (arrow heads). Scale bars 50 μm. B-D. TI was harvested from 12-14 week-old WT or TNF^{ΔARE/+} mice. IECs and LP cells were isolated as described and protein was extracted. B. TfR1 levels were decreased in the LP cells of TNF^{ΔARE/+} mice (inflamed) compared to WT mice (control). Representative SDS-PAGE of Tfr1 of LP cells and IECs (upper) and quantification of TfR1 levels (lower). Values presented as mean±SD, n=4. C. IRP2 levels are differentially activated in IEC and LP of the inflamed terminal ileum in an iron dependent manner. Representative SDS-PGAE gel. Quantification of IRP2 levels. Values presented as mean±SD, n>16. D. Iron regulatory protein (IRP)1 is activated in IECs and LP of the inflamed terminal ileum of TNF^{ΔARE/+} mouse model, in an iron independent manner. Representative gel of an electromobility shift assay of the IEC and LP fractions of the terminal ileum (upper). Quantification of the bands (lower). Values presented as mean±SD, n>4.

FIGS. 2A-B show that an interspecies co-culture model mimics the iron-independent inflammatory activation of IRP1. LPS 200 (ng/ml) was added to co-cultures of EC-MΦ (Raw 264.7) for 24 hours. Cells were harvested and proteins were isolated as described. A. IRP1-RNA activity in macrophages was increased upon LPS induction. Representative autoradiogram showed a significant increase in macrophage IRP1-RNA binding activity (n=4). B. Aconitase activities were decreased in epithelial cells and macrophages. Aconitase activities were detected chromogenically as described. n=4.

FIGS. 3A-D show that LPS stimulation caused a strong increase in macrophage ferritin and a shift in its subunit composition. LPS 200 ng/ml was added to EC-BMDM (bone marrow derived Macrophages) co-cultures as described. A. Iron ferritin increases in macrophages and decreases in epithelial cells during LPS stimulation. EC-BMDM co-cultures were incubated with ⁵⁵Fe-labeled human transferrin as described. LPS was added to the co-culture for indicated times. Iron ferritin was chased in control and LPS stimulated co-cultures. Representative radiograph of lysates separated under native conditions. n=2. B. Macrophage ferritin subunit H was significantly increased after LPS stimulation and the H subunit degradation product decreased significantly after LPS stimulation. L PS was added to EC-BMDM co-cultures for 24 hours. Representative SDS-PAGE of ferritin H (upper) and L (lower) subunits. Under common culture conditions epithelial ferritin was not detected by SDS-PAGE analysis, and the signal was exclusive for macrophage ferritin. Moreover, FtL antibody detects both subunits and distinguishing between both subunits was done according to the molecular weight of each of the ferritin subunits. C. Macrophage ferritin undergoes less degradation during LPS stimulation. EC-BMDM co-cultures were metabolically labeled with ³⁵S as described. Control and LPS stimulated co-cultures were chased for indicated times. Representative pulse-chase radiograph of co-culture lysates after immunoprecipitation with ferritin antibody. n=2. D. Levels of NCOA4 were significantly decreased in inflamed macrophages. The expression levels of epithelial NCOA4 were not significantly changed after LPS stimulation. LPS was added to EC-BMDM (MΦ) co-cultures for different times as indicated. Expression levels were measured by qRT-PCR. n=4.

FIGS. 4A-C show that iron-regulated proteins mimic the inflammatory iron-pattern of reduced epithelial- and increase macrophage-iron. LPS 200 ng/ml was added to EC-MΦ (BMDM or Raw 264.7) co-cultures as described. A. Expression levels of MΦ (BMDM) (left) and epithelial (right) TfR1 showed dynamic changes through the course of LPS stimulation. Expression levels were measured by qRT-PCR. n=4. B-C. LPS was added for 24 hours. B. TfR1 levels were decreased in MΦ (Raw 264.7) and increased in epithelial cells. Representative SDS-PAGE of TfR1. n=12. C. IRP2 levels were decreased in macrophages (BMDM) and increased in epithelial cells. Representative SDS-PAGE of IRP2. n=12.

FIGS. 5A-F show a suggested mechanism of inflammation-mediated activation of IRP1-IRE binding activity. LPS (200 ng/ml) was added to ECs-MΦ co-cultures as indicated A. Expression levels of macrophage (BMDM)-key-proteins associated with iron-sulfur cluster biogenesis decreased significantly during the course of LPS stimulation (upper). Expression levels of epithelial key proteins associated with iron-sulfur cluster biogenesis did not change during the course of LPS stimulation (lower). B. Hif 2α levels were significantly increased after 4 hours of LPS stimulation of EC-MΦ (Raw 264.7). Representative SDS-PAGE gels of subcellular fractions, cytosolic nuclear soluble and nuclear chromatic bound fractions (upper). Protein levels were quantified with Image J (lower), values presented as mean±SD. n=4. * DFO 100 Um for 24 hours. C-E. Co-culture of epithelial cells and macrophages with targeted deletion of IRP1 (MΦ Irp1^{-/-}) didn't restore iron homeostasis in the co-culture upon LPS stimulation. Epithelial cells were co-cultured with BMDM isolated from C57BL/6 mice (MΦ wt) or from IRP^{-/-} on C57BL/6 background mice (MΦ Irp1^{-/-}) as described. LPS 200 ng/ml was added for 24 hours. C. macrophage-FtH was significantly increased in both EC-MΦ wt and EC-MΦ Irp1^{-/-} co-cultures upon LPS stimulation. Representative immunoblot of FtH. n=8. D. The changes in epithelial and macrophage IRP2 levels were not significantly different in EC-MΦ wt and EC-MΦ Irp1^{-/-} co-cultures. Representative blot of IRP2. n=8. E. Aconitase activities were decreased in epithelial cells and macrophages in EC-MΦ wt and EC-MΦ Irp1^{-/-} co-cultures. Representative aconitase in-gel assay. n=4. F. Pre-conditioning of epithelial cells with tempol significantly increased the expression levels of selected epithelial pro-inflammatory markers including epithelial. Caco2 cells were cultured with tempol (3 mM) for 16 hours, cells were then washed, and Raw 264.7 cells were added. When Raw 264.7 cells were adhered, the excess of cells was washed away and LPS was added for 6 hours. Expression levels were measured by qRT-PCR. n=6.

FIGS. 6A-F show that LPS (200 ng/ml) was added to ECs-MΦ (BMDM or Raw 264.7) as described. A. LPS stimulation activated MAP-kinase pathways, ERK 1/2 and p38, and NFkB p65 in EC-MΦ (Raw 264.7) co-cultures. Representative gels showed that the p-ERK 1/2 and p-NFkB p65 levels increased shortly after LPS induction (0.5 h). Phosphorylation of p38 increased only 4 h after LPS stimulation. B. Downstream response of pro-inflammatory cytokines including 118 and ICAM1, CXCL10 in epithelial cells and TNFa, ICAM1, Il1b and Il6 in macrophages (BMDM). Relative expression was evaluated with pre-calibrated primers specific to each species by qRT-PCR. n=4. D. ECs and MΦ response to LPS stimulation is highly augmented when stimulated in co-culture compared to when stimulated individually. LPS was introduced to EC-MΦ (Raw 264.7) or to both cells separated as described. Relative expression was evaluated with pre-calibrated primers specific to each species by qRT-PCR. E. SNAP induces elevated IL-8 mRNA expression levels in Caco-2 cells. IL-8 mRNA levels were evaluated by qRT-PCR. Values presented as mean±SD, n=3. F. SNAP induces intracellular iron decrease via iron secretion. The CPM was determined in the cells (left) and in the medium (right) by a beta-scintillation counter. Values presented as mean±SD, n=6.

FIGS. 7A-C is as follows: A. H rich subunit of ferritin hetero-complex migrates slower than L rich subunit of ferritin hetero-complex. Upper: HEK293T cells transfected with indicated constructs. Coomassie- (left) and Perls-DAB iron-stain (right) of lysates separated on native-PAGE. 1:1 FTH:FTL transfection appears as a defined smear, suggesting assembly to diverse subunit combinations. Endogenous ferritin is below detection levels in HEK293T cells (left lane). Lower: Protein extracted from tissues harvested from C57BL/6 mice was separated on native gel. B. Expression levels of ferritin subunits were differentially changed in macrophage and epithelial cells during the course of inflammation. LPS (200 ng/ml) was added to EC-BMDM (MΦ) co-cultures for different times as indicated. Expression levels were measured by qRT-PCR. n=4. C. The ratio between H and L ferritin in the complex after LPS stimulation was 3 times more than the control. Relative protein levels were evaluated via ImageJ. n=4

FIGS. 8A-E are as follows: A. The expression levels of the epithelial iron homeostasis proteins DMT1 and Fpn increased in correlation with the increase in Hif2α levels. Expression levels of macrophage DMT1 and Fpn decreased through the course of LPS stimulation. LPS (200 ng/ml) was added to ECs-BMDM co-cultures for different times as indicated. Expression levels were measured by qRT-PCR. n=4. B-D. Knock out of IRP1 in macrophages did not affect the inflammatory response of the co-culture after LPS stimulation. B. IL8 increase after LPS stimulation was not significantly different in EC-MΦ wt compared to EC-MΦ Irp1^{-/-} co-cultures.IL8 concentrations in the medium were evaluated using IL8 ELISA kit as per the manufacturer's instructions. n=6. C. Selected macrophage pro-inflammatory activators including Tnfα, Icam1 and Il6 expression levels were not significantly different in EC-MΦ wt compared to EC-MΦ Irp1^{-/-} co-cultures. n=2. * Expression calculated relative to the control WT co-culture. ** Expression calculated relative to WT co-culture stimulated with 50 ng/ml LPS. Nitrite concentrations after LPS stimulation were not significantly different in EC-MΦ wt compared to EC-MΦ Irp1^{-/-} co-cultures. D. Nitrite concentrations in the medium were assessed by Griess assay. n=6. E. Activation of IRP1 in the co-culture induces a trend towards a pro-inflammatory response. ECs-Raw 264.7 cells (MΦ) were co-cultured as previously described. Tempol (3 mM) was then added for different times as indicated. Representative aconitase in-gel activity blot (upper) showed decreased aconitase activities in a direct correlation with the incubation time. The transcription levels of the pro-inflammatory markers showed an increased trend that was not statistically significant, upon IRP1 activation with tempol (lower). Expression levels were evaluated by RTqPCR.

FIG. 9 is a schematic representation of the involvement of IRP1 in the self-accelerating inflammatory process. Absence of epithelial IRP1 prevents inflammation exacerbation by discontinuing the iron flux.

### DESCRIPTION OF SPECIFIC EMBODIMENTS OF THE INVENTION

The present invention, in some embodiments thereof, relates to a model for analyzing inflammation and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Inflammatory diseases, such as inflammatory bowel diseases (IBD), are complex disorders that are treated mostly by suppressing activated inflammatory pathways of the immune system. Recently, mitochondrial defects and impaired energy homeostasis were identified as possible drivers of the inflammation, suggesting an involvement of iron homeostasis in the pathophysiology of IBD and other inflammatory conditions. In the inflamed tissue of IBD-patients, iron accumulation was demonstrated, but neither a mechanism for the accumulation nor its function in the course of inflammation were known. Clearly a model for inflammation and specifically gastrointestinal inflammation would be of great value in assessing inflammation and its modulators.

Whilst conceiving embodiments of the invention, the present inventor observed that inflamed epithelial cells take on an iron deprived phenotype, while inflamed immune cells take on an iron loaded phenotype, yet IRP1-RNA-binding activity was elevated in both cell-types. However, the opposing inflammatory regulation of iron homeostasis that included iron increase in macrophages and iron decrease in epithelial cells in the same tissue makes it very difficult to study a mechanism for this redistribution of iron on a molecular level. Currently available co-culture models which aim at deciphering the inflammatory process do not mimic the inflammatory iron pattern (Antunes et al. 2013, supra; and Tanoue et al. 2008, supra).

Thus, whilst further reducing the present invention to practice, the present inventors devised a novel model for inflammation. This model is based on a co-culture of tissue and immune cells of different species, said cells being in direct contact. Only when epithelial cells and macrophages are in direct contact, the inflammatory iron homeostasis is established including the iron and ferritin distribution. Indeed, as shown hereinbelow and in the Examples section which follows, the present inventors observed similar iron related phenotypes between the in vitro model of some embodiments of the invention and an in vivo IBD model. It will be appreciated that the use of an interspecies co-culture, has many advantages: it enables to differentially analyze iron related proteins such as TfR1, IRP1 and 2, from epithelial and macrophage origin. Indeed, in this way the present inventors could show, that TfR1 and IRP2 were regulated by the iron state of the specific cell-types (increased in epithelial cells and decreased in macrophages), in response to the induction of inflammation in the co-culture. In addition, the present inventors could show that IRP1 is activated in both cell types, and does not respond primarily to the cellular iron state, but much more importantly seems to be the dominant regulator of inflammatory iron homeostasis.

The interspecies co-culture was also used to show the inflammation mediated changes in ferritin: primarily a significant increase of macrophage ferritin and ferritin iron in response to the inflammation and also a significant shift in the ferritin subunit composition.

Thus, the present model can be used to differentially and accurately decipher the effect of different stimuli on iron accumulation/distribution in tissue and immune cells, by the mere use of different species. It is also simple and cost-effective to execute, as the cells are in direct contact, negating the need for a barrier as in previous models. As such the present model can be used as a valuable tool in screening of various agents including, drugs, foods and beverages or any other potential hazard.

Thus, according to an aspect of the invention there is provided an in vitro culture model for inflammation comprising tissue cells and immune cells, wherein the tissue cells and immune cells are of different species and are in direct contact.

As used herein “inflammation” refers to an in vivo physical condition in which a part of tissue in a body may become reddened, swollen (enlarged), or damaged (ulcerated) especially as a reaction to injury or an irritant. Areas of inflammation can have increased blood flow and capillary permeability, i.e. changes in endothelial cells lining capillaries resulting in capillary dilation and leukocyte infiltration into the irritated and/or inflamed tissues, along with activated immune cells, including white blood cells, leukocytes, lymphocytes, etc., including substances produced by activated immune cells. Inflammation may occur suddenly (acute) or gradually over time (chronic). Inflammation may be local, i.e. in one location as a “patch” or “spot” or may be in several areas as numerous patches, including ulcers, or contiguous involving a large area of tissue. Inflammation may be limited to epithelial regions and underlying lamina propria (for example, mucosal areas), or may extend to the submucosa, or extend to the muscularis propria and may further extent to the outermost layer, adventitia, in contact with other parts of the body.

According to a specific embodiment, in the context of the model, inflammation refers to a physiological condition in vitro, which is characterized by molecular parameters that are characteristic of inflammation.

As used herein “tissue cells” refer to solid tissue cells.

Examples of such cells include, but are not limited to, epithelial cells, connective tissue cells, blood vessels forming cells (smooth muscle cells, endothelial cells), stromal cells, hepatocytes, neural cells (e.g., neuronal or glial cells, e.g., astrocytes, oligodendrocytes), cardiac cells, muscle cells, bone cells, cartilage cells, ligand/tendon cells, pancreatic cells, ophthalmic cells, renal cells and more.

According to a specific embodiment, the tissue cells are enriched for non-immune cells. That is, when isolated, the tissue biopsy is washed to remove residential immune cells. However, the specimen may still comprise immune cells of the species of the tissue cells but to a much lower degree than physiologically present. This for example, the “tissue cells” can be a pure population, such as when making use of a cell line, or a mixed population of cells which may comprise for example: epithelial cells, fibroblasts and endothelial cells.

According to a specific embodiment, the tissue cells are of the gastrointestinal tract.

According to a specific embodiment, the tissue cells are colon epithelial cells.

As used herein “immune cells” refer to cells which constitute the tissue immune system. According to a specific embodiment the immune cells are also referred to as tissue-resident immune cells. Tissue-resident immune cells span both myeloid and lymphoid cell lineages, have been found in multiple tissues, and play integral roles at all stages of the immune response, specifically, according to the present teachings, they maintaining homeostasis to responding to infectious challenges to resolution of inflammation to tissue repair.

According to a specific embodiment, the immune cells are Lamina Propria (LP) cells.

As used herein, the terms “lamina propria-derived cells” and “LP-derived cells” refer to cells used in the context of specific tissues (e.g. mucosal tissues), including but not limited to stromal cells, fibroblasts, and immune cells (including resident immune cells or immune cells that may be transiently present in said tissues).

According to a specific embodiment the LP cells shall include all cell types that one could derive from lamina propria. In one embodiment, LP-derived cells are isolated from specific tissues (e.g. mucosal tissues). LP-derived cells are not limited to mucosal tissues, as they may be isolated from tissues extending into mucosal areas, for example, cells in stromal areas.

According to a specific embodiment, the immune cells include but are not limited to B cells, T cells, dendritic cells, monocytes, macrophages, innate lymphoid cells, or combinations thereof. According to a specific embodiment, the immune cells are macrophages. According to a specific embodiment, the macrophages are differentiated in vitro from monocytes, as the macrophages, as well as those cells mentioned can be derived from lamina propria.

According to a specific embodiment, the immune cells are bone marrow-derived.

According to a specific embodiment, the immune cells are from the peripheral blood.

According to some embodiments, the cells can be freshly isolated cells.

According to some embodiments, the cells can be primary cells.

According to a specific embodiment, the cells are of a cell line: as shown in the examples section, the model can use Caco2 cells for the tissue cells and Raw 264.7 macrophages.

According to some embodiments, the cells can be of a healthy tissue (e.g., non-inflamed).

According to some embodiments, the cells can be diseased, e.g., inflamed cells, such as of IBD, rheumatoid arthritis, psoriasis, cancer, fatty liver diseases, diabetes, obesity.

According to a specific embodiment, both immune cells are inflamed cells are healthy.

According to a specific embodiment, both immune cells are inflamed cells are diseased.

According to a specific embodiment, the immune cells are inflamed cells and the tissue cells are healthy.

According to a specific embodiment, the tissue cells are inflamed cells and the immune cells are healthy.

Any of the cells may be used directly after isolation or undergo culture to expand cell numbers prior to use. The cells may undergo isolation techniques before or after culturing or freezing. In other embodiments, the cells may be cryopreserved (frozen) prior to use.

The tissue cells and/or the immune cells can include one or more cell types. For example, the tissue cells may include epithelial cells or epithelial+fibroblasts; and the immune cells may include macrophages, monocytes, dendritic cells alone or in combinations of 2-3 for example.

As used herein “different species” refer to a pair (or more when more than 2 cells types are used) of species in which an inflammation parameter can be distinguished based on its different phenotype in the different species. As will be further described hereinbelow, the different phenotype can be for example, different molecular weight (MW), different post translational modification (e.g., phosphorylation), different cellular distribution, different sequence (DNA, RNA or Protein), different epi-genetics, different splice variant expression, different second messengers.

According to a specific embodiment, the tissue cells are human cells.

According to a specific embodiment, the tissue cells are non-human cells.

According to a specific embodiment, the immune cells are human cells.

According to a specific embodiment, the immune cells are non-human cells.

According to a specific embodiment, the tissue cells are adherent.

According to a specific embodiment, the tissue cells are non-adherent.

According to a specific embodiment, the tissue cells are human cells and the immune cells are non-human cells.

According to a specific embodiment, the immune cells are mammalian cells, such as rodent cells, e.g., murine, rat, avian cells or of an aquatic organism such as fish or crustaceans.

As used herein “direct contact” means that at least some of the tissue cells and immune cells are in contact with no barrier between them. Direct contact also means that all the secretome of each of the immune cells and the tissue cells can be in contact with the reciprocal cells, for instance, the secretome of the immune cells can be in contact with the tissue cells and vice a versa under the settings of the co-culture of the invention.

Culturing can be done in any culturing dish, at any scale, be it a simple petri dish, multi-well dish or a sophisticated lab on chip apparatus. The skilled artisan would know which medium, factors, sera, atmosphere and other culturing conditions to use, such as oxygen 3-12%, e.g., 6%.

Culturing can be static or perfused (such as via a channel).

The ratio of immune cells to tissue cells of the different species can change dependent on the intended use. According to a specific embodiment, the ratio is between 10:1 to 1:10, 8:1 to 1:8, 6:1 to 1:6, 4:1 and 1:4, 3:1 to 1:3, 2:1 to 1:2 or 1:1.

The immune cells and tissue cells (of the different species) can be seeded simultaneously or sequentially, as long as a sustainable co-culture of both species is established, i.e., both cell populations are viable when the co-culture is used for assaying a particular agent. According to a specific embodiment, the culture persists for at least 2, 4, 6, 8, 10, 12, 14, 16, 18, 24, 26, 48, 72, 96 hours and for example no more than 7, 5 or 4 days.

Thus, according to a specific embodiment, there is provided an in vitro method of assaying inflammation in culture, the method comprising:
(a) contacting the model as described above with a test agent; and
(b) analysing an inflammation parameter in said tissue cells and/or immune cells.

As used herein “test agent” refers to any chemical or biological substance at any state or a physical condition. The test agent can be analysed for research, clinical or commercial purposes without intending to be limited by the use. Examples of test agents include but are not limited to a small molecule, an antibody, a peptide, a carbohydrate, a fatty acid, a synthetic substance, a natural substance, a mimetic, a library or portion thereof or combinations of same.

The agent can be an “irritant”, i.e., an agent that induces the state of irritation such as of an epithelial lining, for example, a bacterial toxin or an allergen that causes activation of resident mononuclear white blood cells, leukocytes, lymphocytes, etc. in the lamina propria (in vivo), lamina propria-derived cells (in vitro), or actual damage to epithelial cells, in vivo or in vitro, that in turn triggers activation of resident immune cells any of which may induce irritation. As used herein, the term “irritation” refers to initiation of inflammation. By way of example only, this may be due to an allergy or damage to epithelial cells in the lining of the gastrointestinal system.

The amount and time of exposure to the test agent will be determined by the skilled artisan. The assay can be replicated, duplicated and essentially repeated as needed. A control without the test agent can be included in the settings too. Another control would be, if such desired, in the presence of a known inflammatory inducing agent and/or a known innocuous agent, which does not elicit inflammation. Other controls are envisaged and will depend on the setting of the assay.

As mentioned, an inflammation parameter is determined. It can be determined in real time, or following a predetermined time in culture. It can be determined on a fresh co-culture or following storage, e.g., in 4° C. to −20° C.

According to a specific embodiment, analyzing is done at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 hours or more e.g., 48 hours and/or up to 4 days for instance.

It will be appreciated that starting the analysis after at least 4 hours may ensure in some embodiments and at least in some parameters acquiring a true inflammatory phenotype, whereby epithelial cells have reduced iron levels and the immune cells have accumulated it. As shown in the Examples section which follows, consistent with the iron accumulation in the macrophages and the iron decrease in the IECs of the TNF^{ΔARE} mice, TfR1 mRNA levels decreased 24 hours after LPS exposure in the macrophages and increased in the Caco2 cells. The TfR1 decrease in macrophages was preceded by an increase in TfR1 transcripts, two hours after LPS stimulation supporting an early inflammation mediated increase in iron import that leads to the iron accumulation in the macrophages.

According to a specific embodiment, the inflammation parameter is associated with iron homeostasis.

As used herein “iron homeostasis” refers to cellular iron homeostasis. In its simplest manifestation it relates to the labile iron pool (LIP) and to cellular iron stores, i.e., cellular iron amount or cellular or sub-cellular distribution. As the present inventors have shown, during inflammation there is an increase in iron stores in immune cells, e.g., macrophages, relatively to such cells when non-inflamed. This may be accompanied by alteration in LIP and iron stores in tissue cells. For example, in epithelial cells in organs of barrier function e.g., GI, testical, breast, brain, there is a decrease in iron stores as compared to the same cells when not inflamed. Without being bound by theory, it is suggested that epithelial iron efflux causes the immune cell iron accumulation. In other tissue cell types for instance, fibroblasts, neural, hepatic cells (e.g., hepatocytes) there may be an increase in iron stores as compared to the same cells when not inflamed.

According to a specific embodiment, the inflammation parameter associated with iron homeostasis is iron amount or an indicator thereof in said tissue cells and/or immune cells.

In some embodiments, an indication of inflammation is determined by the iron amount. When the iron amount is increased in immune cells with respect to non-inflamed immune cells of the same type an inflammation is indicated.

Alternatively or additionally, when the iron amount is altered the said tissue cells with respect to non-inflamed tissue cells of the same type, inflammation is indicated.

As used herein “altered” is an increase or decrease with respect to control as described above, e.g., with respect to non-inflamed tissue cells of the same type. The alteration is statistically significant, e.g., Two tailed student t-test.

As used herein “increase” refers to an increase in activity or expression (protein or mRNA) of at least 10%, 20%, 30%, 40%, 50%, 60%, 70%, 80%, 90%, 2 fold, 5 fold, 10 fold or more, as compared to control, e.g., non-inflamed cells of the same type.

As used herein “decrease” refers to a decrease in activity or expression (protein or mRNA) of at least 10%, 20%, 30%, 40%, 50%, 60%, 70%, 80%, 90%, 2 fold, 5 fold, 10 fold or more, as compared to control, e.g., non-inflamed cells of the same type.

It will be appreciated that the exact phenotype, i.e., increase or decrease is determined according to the tissue origin of the tissue cells as well as the tissue type. For example, it is suggested that in epithelial cells of tissue having barrier functions, such as GI tissue, e.g., intestinal tissue, testical tissue, blood brain barrier, mammary barrier and the like, there is a decrease in iron amount in said tissue cells with respect to control, e.g., as described above, e.g., with respect to non-inflamed tissue cells of the same type.

However, for other cells of the same tissue origin, a different phenotype may exist. For example, it is contemplated that for fibroblasts an increase in iron amount in said tissue cells with respect to control, e.g., as described above, e.g., with respect to non-inflamed tissue cells of the same type, will be indicative of inflammation.

There are numerous methods for determining cellular iron, and the skilled artisan would be able to select. Some examples include, in a non-limiting manner, colorimetric assays such as Ferrozine assay and Bathophenantroline assay, and in live cells there are measurements for the LIP-labile iron pool by Ferroorange or Calcein-AM.

According to an additional or an alternative embodiment, the inflammation parameter associated with iron homeostasis is an iron-regulated gene.

As used herein “an iron regulated gene” refers to a gene which protein product activity or amount is regulated by iron in a direct or indirect manner (for instance affecting the level of RNA or protein conformation or protein breakdown).

Examples of iron regulated genes include, but are not limited to ferritin, Transferrin Receptor (TfR)1, NCOA4, IRP1 or IRP2.

As mentioned, iron homeostasis is tightly regulated, at the cellular level, mainly by the iron regulatory proteins (IRPs)1 and 2. When cellular iron is low, IRPs bind with high affinity to RNA motifs known as iron-responsive elements (IREs), which are found in numerous transcripts, mostly related to iron homeostasis. IRP2 is degraded by high iron, oxygen and ROS/RNS levels. IRP1 is a bifunctional protein changing conformation from a cytosolic iron sulfur-cluster containing aconitase (cAco) at high cellular iron to an RNA (IRE)-binding apoprotein, when cellular iron is low. The present inventors have found that the RNA binding activity of IRP1 is increased during inflammation in both immune cells and tissue cells, and does not respond primarily to the cellular iron state, but much more importantly seems to be the dominant regulator of inflammatory iron homeostasis.

Thus, when the amount of cellular iron is altered, the RNA binding activity will be more dominant than its aconitase activity. Each of the RNA binding activity (increase) and the aconitase activity (decrease) can be used as an indication for inflammation.

As used herein “ACO1” or “iron regulatory protein (IRP)1” abbreviated as “IRP1” refers to the mRNA or protein product of gene symbol ACO1, E.C. No.: 4.2.1.3.

The human protein sequence is provided in NM_004136. Other homologs such as of the mouse are provided in NM_007386.2.

As used herein “iron regulatory protein (IRP)2” abbreviated as “IRP2” refers to the mRNA or protein product of the IREB2 gene.

Refseq RNA numbers for the human gene include NM_001320942.2, NM_001320943.2, NM_001354994.2, NM_004136.4. For orthologs, see for example, NM_022863.2 or NP_074054.2, products of the rat gene.

According to a specific embodiment, the iron-regulated protein is IRP2, wherein inflammation is indicated by a decrease in the IRP2 expression in the immune cells, wherein said decrease is with respect to control, e.g., non-inflamed cells of the same type.

Thus, according to an embodiment of the invention, the inflammation parameter associated with iron homeostasis is a decrease in cellular or mitochondrial aconitase activity of IRP1 in the immune cells and the tissue cells, wherein said decrease is with respect to control non-inflamed cells of the same type.

Methods of measuring aconitase activity or amount are well known in the art.

Mitochondrial and cytosolic aconitase activities can be assayed in cell lysates after electrophoretic separation as described in W. H. Tong, T. A. Rouault, Functions of mitochondrial ISCU and cytosolic ISCU in mammalian iron-sulfur cluster biogenesis and iron homeostasis. Cell metabolism 3, 199-210 (2006). Protein concentrations are determined using the Bradford assay.

The aconitase activity can be detected chromogenically by incubating the gels with the substrate e.g., cis-aconitic acid under conditions which allow in gel catalytic activity. This allows species specific detection of the activity of IRP1.

According to another embodiment, the iron regulated gene is ferritin.

The interspecific model presented herein allows to follow inflammation-mediated changes in ferritin. The present inventors found not only a significant increase of macrophage ferritin and ferritin iron in response to the inflammation but also a significant shift in the ferritin subunit composition. Likewise, the levels of NCOA4 were decreased in inflamed macrophages.

As used herein “Nuclear receptor coactivator 4” abbreviated as “NCOA4” refers to the mRNA or protein product of the gene having the symbol NCOA4. According to a specific embodiment, the inflammation parameter associated with iron homeostasis is a decrease in NCOA4 in immune cell, wherein the decrease is with respect to control, e.g., non-inflamed cells of the same type.

As used herein “ferritin” refers to the mRNA or protein product of ferritin. The protein has 2 major subunits: FTL (e.g., for human NM_000146, for the mouse NM_010240.2) and FTH1 (e.g., for human NM_002032. FOR THE MOUSE, PLEASE BC012314). Typically, the amount of ferritin or subunits thereof can be determined by protein or mRNA detection assays, such as with different primers and labels for each species subunit, as further described hereinbelow.

Thus, according to an embodiment of the invention, the iron-related protein is ferritin and analyzing is by analyzing H-subunit (FTH1) and L-subunit (FTL) thereof, wherein inflammation is indicated wherein FTH1:FTL protein ratio above 1 preferably above 2 in the immune cells. Likewise, FTH1:FTL protein ratio, can be altered (e.g., increased or decreased) in the tissue cells as compared to control e.g., non-inflamed cells of the same type. For example the ratio can be above 1 (e.g., above 2) in tissue cells is they accumulate iron, or below 1 if the iron level in the tissue cells decreases.

According to another embodiment, the iron-regulated gene is ferritin and the analysing is by analysing ferritin-iron complex in said immune cells and wherein inflammation is indicated by an increase in said ferritin-iron complex in said immune cells, wherein said increase is with respect to control non-inflamed cells of the same type.

The ferritin-iron complex can be determined by pulse-chase with transferrin-⁵⁵Fe. Cells are incubated with transferrin-⁵⁵Fe for a given time period (1-16 hours-pulse), washed and incubated for up to 72 hours in regular unlabeled medium (chase). Incorporation of ⁵⁵Fe into ferritin is evaluated by separating cell proteins on native Polyacrylamide gel electrophoresis (PAGE) and visualizing ⁵⁵Fe in ferritin.

In an alternative assay, the visualization of iron in ferritin is envisaged, after native PAGE by iron-stain, namely Perls/Prussian blue enhanced by 3,3′-Diaminobenzidine (DAB).

According to another embodiment, the iron-regulated protein is TfR1, wherein inflammation is indicated by a decrease in said TfR1 expression in the immune cells, wherein said decrease is with respect to control non-inflamed cells of the same type.

As used herein “Transferrin Receptor 1” abbreviated as “TfR1” refers to the mRNA or protein product of the TFRC gene. Exemplary products of the human genes are provided in NP_035768, NP_001344227, NP_001121620, NP_001300894 and NP_003225. For orthologs see for example of the mouse: NM_011638.4 and NP_035768.1.

According to some embodiment, the detection of TfR1 is determined by a protein assay, for example, an antibody-based assay, such as a Western blot. Other methods for determining expression (mRNA or protein) or activity can be determined as known in the art and summarized below. Alternatively or additionally, transferrin receptor activity can be quantified. For example, TfR1 can be quantified by binding fluorescent transferrin and measuring binding/internalization by flow cytometry/FACS.

Also, iron delivery can be evaluated directly and indirectly by giving transferrin-Fe and measuring cellular iron or the response of iron sensing proteins.

According to a specific embodiment, the inflammation parameter associated with iron homeostasis is a protein involved in Fe—S cluster assembly and wherein inflammation is indicated when a decrease in the immune cells is indicated, wherein a decrease is with respect to control, e.g., non-inflamed cells of the same type.

As used herein “a protein involved in Fe—S cluster assembly” refers to an iron-sulfur cluster biogenesis protein. There are numerous such enzymes.

Mitochondrial Fe—S clustering or iron-sulfur (ISC) biogenesis has two functions: (1) to synthesize functional clusters in the mitochondria, and (2) to provide an essential precursor to the CIA pathway via the inner membrane exporter ABCB7. Briefly, persulfide ions are generated by cysteine desulfurase (NFS1). Iron and sulfide ions are then delivered to a scaffold protein ISCU2 to form an initial 2Fe-2S cluster. Then chaperones transfer this 2Fe-2S cluster to a glutaredoxin, which subsequently delivers the 2Fe-2S cluster to the target protein or to the next Tron-sulfur assembly protein (ISA) complex. The ISA complex can condense two 2Fe-2S clusters into one 4Fe-4S center. Major components involved in the ISC pathway are shown in Table A. In sum, 2Fe-2S and 4Fe-4S proteins are made differently, with de novo 2Fe-2S clusters forming on the ISCU scaffold and 4Fe-4S clusters forming subsequently in a downstream step utilizing the ISA complex of proteins.

TABLE A
(taken from Shi et al. Front. Cell Dev. Biol., 30 Sep. 2021)

| Complex | Human | Yeast | Function | Location |
|---|---|---|---|---|
| ISCU | ISCUs | Isu1/Isu2 | Scaffold protein | Mitochondria, |
| complex | | | | cytoplasm and |
| | | | | nucleus |
| | NFS1 | Nfs1 | Cysteine | Mitochondria, |
| | | | desulfurase | cytoplasm and |
| | | | provides the sulfur | nucleus |
| | ISD11 | Isd11 | Stabilize binding | Mitochondria |
| | | | partner of NFS1 | and nucleus |
| | ACPM | Acp1 | Bind to and | Mitochondria |
| | | | stabilize Isd11 | |
| | Ferredoxin | Yah1 | Electron donor | Mitochondria |
| | Frataxin | Yfh1 | Regulates NFS1 | Cytoplasm and |
| | | | activity | nucleus |
| Cluster | HSPA9 | Ssq1 | Co-chaperone of | Mitochondria |
| transfer | | | GLRX6 | and nucleus |
| complex | | | | |
| | HSC20 | Jac1 | Co-chaperone of | Mitochondria, |
| | | | GLRX5 | cytoplasm and |
| | | | | nucleus |
| | GLRX5 | Grx5 | Transfers 2Fe—2S | Mitochondria |
| | | | clusters to client | |
| | | | proteins | |
| | ABCB7 | Atm1 | Exports Fe—S | Mitochondria |
| | | | clusters from | |
| | | | mitochondria | |
| | ALR | Erv1 | Exports Fe—S | Mitochondria |
| | | | clusters from | and cytoplasm |
| | | | mitochondria | |
| ISA | ISCA1 | Isa1 | Assemble 4Fe—4S | Mitochondria |
| complex | | | clusters | |
| | ISCA2 | Isa2 | Assemble 4Fe—4S | Mitochondria |
| | | | clusters | |
| | IBA57 | Iba57 | Assemble 4Fe—4S | Mitochondria |
| | | | clusters | |
| | NFU1 | Nfu1 | Transfer 4Fe—4S | Mitochondria |
| | | | clusters to target | |
| | | | proteins | |

According to a specific embodiment, the protein involved in Fe—S cluster assembly is Nfs1 or IscU1.

As used herein “Nfs1” or “NFS1 Cysteine Desulfurase” refers to the mRNA or protein product of the gene having the symbol NFS1. Exemplary sequence for the human protein is in Refseq NM_001198989 and an exemplary sequence for the mouse gene is NM_010911.

As used herein “Iron-Sulfur Cluster Assembly Enzyme” abbreviated as “IscU1” refers to the mRNA or protein product of the gene having the symbol ISCU1. Exemplary sequences for the human sequences are provided in NM_001301140.1, NM_001301141.1, NP_001288069.1 and NP_001288070.1. Exemplary sequences for the mouse orthologs NM_025526.4 and NP_079802.1

As shown in the Examples section which follows, epithelial cells of an intestinal tissue have a decreased iron amount or an indicator thereof (such as described above, an iron regulated gene) with respect to non-inflamed epithelial cells of the same type, and optionally wherein the tissue comprises an epithelial barrier (e.g., of the GI, choroid plexus, testical, breast).

In other tissue cells and tissues the phenotype can be similar to that of the immune cells such as in fibroblasts, hepatocytes, neurons, osteoblasts or others.

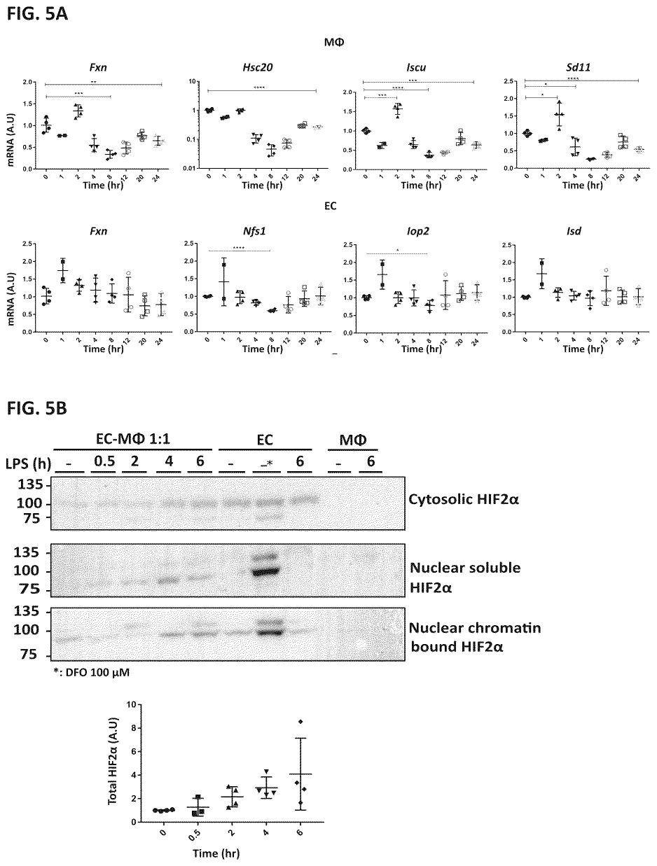

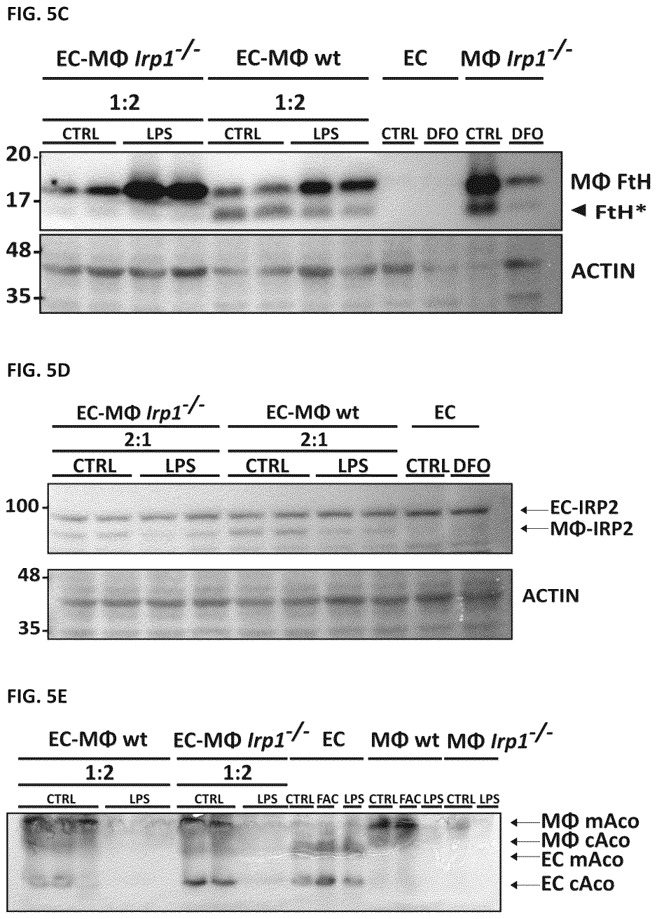

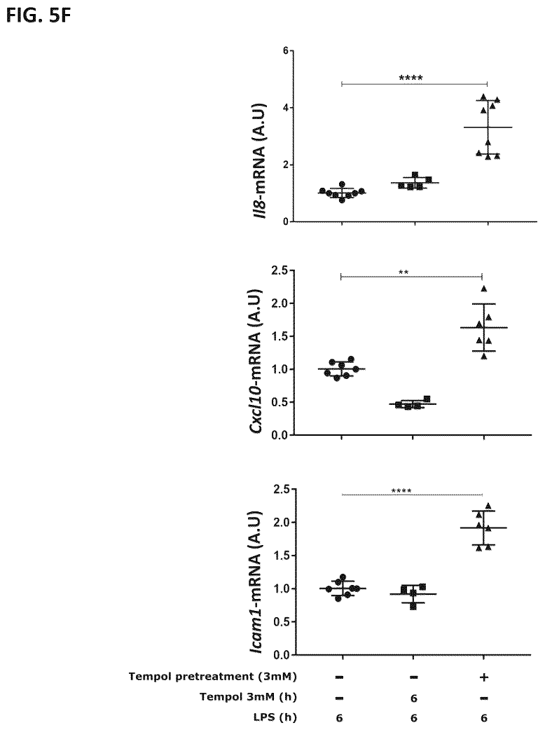

According to a specific embodiment, the inflammatory parameter is HIF2α, whereby HIF2α is increased in inflammation in both immune and epithelial cells. HIF2α is a major regulator of iron uptake through epithelial cells. Both cytosolic and nuclear HIF2α are increased in response to inflammation (FIG. 5B). In line with the HIF2α activation, both, the apical iron importer DMT1 and the basolateral iron exporter FPN increased gradually thereby promoting both apical Fe-influx and basolateral Fe-efflux of the epithelial cells which further supports the epithelial iron-deficiency and consequently an increase in IRP1-RNA binding activity.

Alternatively or additionally, the inflammation parameter is a canonic inflammatory parameter, such as pro or anti-inflammatory cytokine or lymphokine.

Thus, according to specific embodiment, the inflammation parameter is a pro-inflammatory cytokine and/or an anti-inflammatory cytokine, wherein an increase in the pro-inflammatory cytokine and/or a decrease in anti-inflammatory cytokine in the immune cells and/or tissue cells is indicative of inflammation, wherein the increase or decrease is with respect to control non-inflamed cells of the same type.

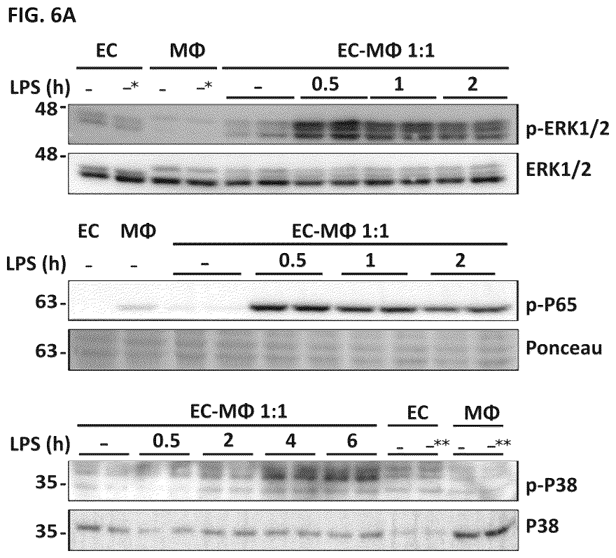

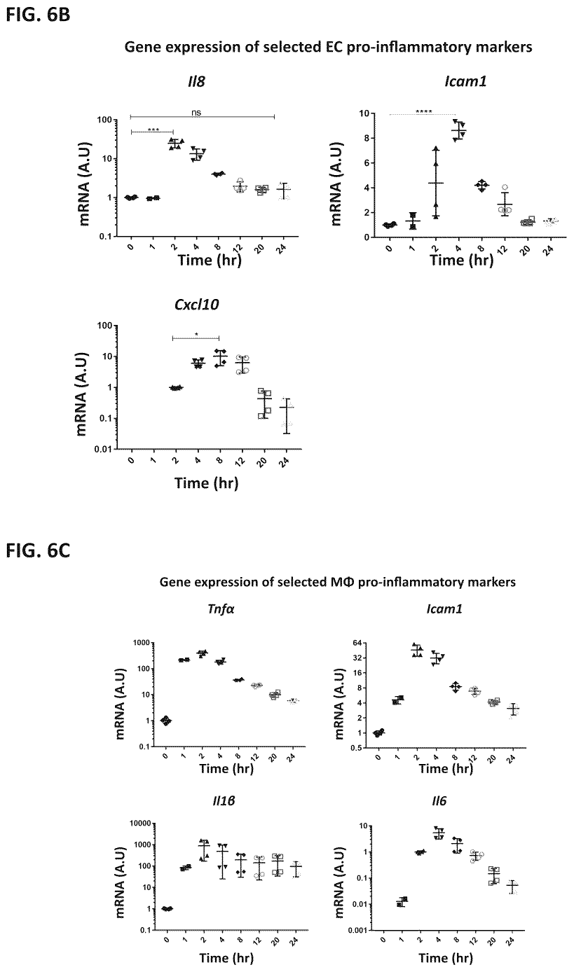

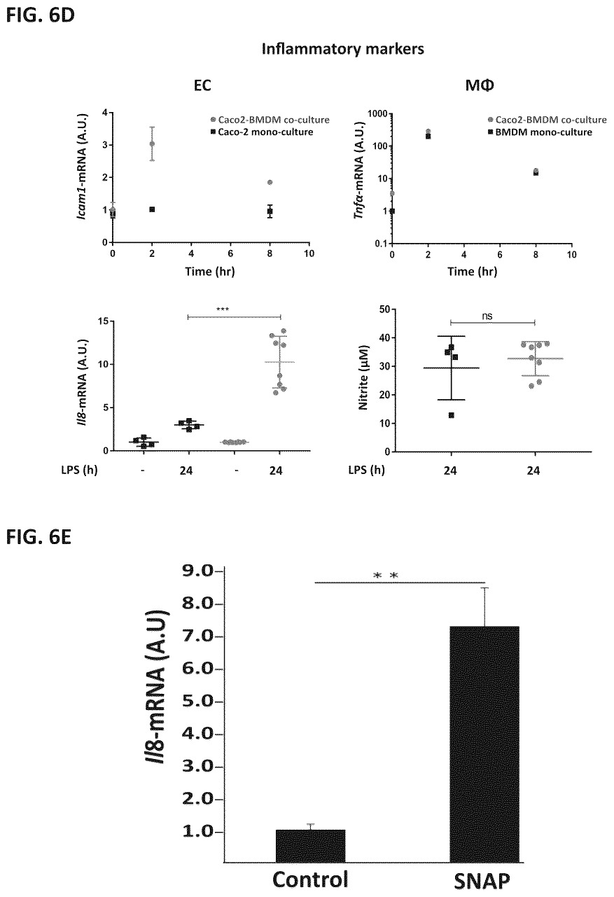

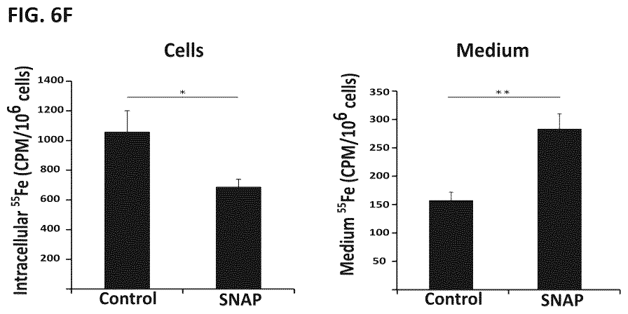

As shown in the Examples section which follows, when an inflammatory agent was added to the co-culture (FIGS. 6A-F) activation of MAP-kinase pathways, ERK 1/2 and p38, and NFkB p65 was evident in both cell types, immune and tissue cells. Phosphorylation of p38 increased and downstream response of pro-inflammatory cytokines including Il8 and ICAM1, CXCL10 in epithelial cells and TNFa, ICAM1, Il1b and Il6 was evident in macrophages.

Thus, according to an embodiment of the invention, the inflammation parameter is a signalling pathway associated with inflammation.

According to a specific embodiment, the signalling is a MAP-kinase pathway, wherein an increase in signalling via the MAP-kinase pathway n the immune cells and/or tissue cells is indicative of inflammation, wherein the increase is with respect to control non-inflamed cells of the same type.

Three main pathways, NF-1B, MAPK, and JAK-STAT, play major roles in inflammation, and dysregulation of one or more of these pathways may lead to inflammation-associated disease. These are well known in the art and reviewed in Newton and Dixit Cold Spring Harb Perspect Biol. 2012 March; 4(3): a006049, which is hereby incorporated by reference.

As used herein “an anti inflammatory cytokine” refers to an inflammatory cytokine or proinflammatory cytokine which is a type of signaling molecule that is secreted from immune cells like helper T cells and macrophages, and other cell types that promote inflammation. Examples include, but are not limited to

As used herein “an anti-inflammatory cytokine” refers to immunoregulatory molecules that control the pro-inflammatory cytokine response. Cytokines act in concert with specific cytokine inhibitors and soluble cytokine receptors to regulate the human immune response.

TABLE B
Summary of selected cytokines and their functions (Turner et
al. Volume 1843, Issue 11, November 2014, Pages 2563-2582)

| | | Main | | | Major |
|---|---|---|---|---|---|
| Cytokine | Classification | Sources | Receptor | Target Cell | Function |
| Erythropoietin | | Endothelium | EpoR | Stem cells | Red blood cell |
| | | | | | production |
| G-CSF | Pro- | Fibroblasts, | CD114 | Stem cells in | Granulocyte |
| | inflammatory | endothelium | | BM | production |
| GM-CSF | Adaptive | T cells, | CD116, | Stem cells | Growth and |
| | immunity | macrophages, | CDw131 | | differentiation |
| | | fibroblasts | | | of monocytes, |
| | | | | | and eosinophil, |
| | | | | | granulocytes |
| | | | | | production |
| IL-1 | Pro- | Macrophages, | CD121a | B cells, NK | Pyrogenic, |
| | inflammatory | B cells, DCs | | cells, T-cells | pro- |
| | | | | | inflammatory, |
| | | | | | proliferation |
| | | | | | and |
| | | | | | differentiation, |
| | | | | | BM cell |
| | | | | | proliferation |
| IL-2 | Adaptive | Th1 cells | CD25 | Activated T | Proliferation of |
| | immunity | | | and B cells, | B cells, |
| | | | | NK cells | activated T |
| | | | | | cells, NK cell |
| | | | | | function |
| IL-3 | Adaptive | T cells | CD123, | Stem cells | Hematopoietic |
| | immunity | | CDw131 | | precursor |
| | | | | | proliferation |
| | | | | | and |
| | | | | | differentiation |
| IL-4 | Adaptive | Th Cells | CD124 | B cell, T cell, | Proliferation of |
| | immunity | | | macrophages | B and |
| | | | | | cytotoxic T |
| | | | | | cells, enhances |
| | | | | | MHC class II |
| | | | | | expression, |
| | | | | | stimulates IgG |
| | | | | | and IgE |
| | | | | | production |
| IL-5 | Adaptive | Th2 Cells and | CDw125, | Eosinophils, | B-cell |
| | immunity | mast cells | 131 | B-cells | proliferation |
| | | | | | and |
| | | | | | maturation, |
| | | | | | stimulates IgA |
| | | | | | and IgM |
| | | | | | production |
| IL-6 | Pro- | Th Cells, | CD126, | B-cells, | B-cell |
| | inflammatory | macrophages, | 130 | plasma cells | differentiation |
| | | fibroblasts | | | |
| IL-7 | Adaptive | BM stromal | CD127 | Stem cells | B and T cell |
| | immunity | cells, | | | growth factor |
| | | epithelial | | | |
| | | cells | | | |
| IL-8 | Pro- | Macrophages | IL-8R | Neutrophils | Chemotaxis for |
| | inflammatory | | | | neutrophils and |
| | | | | | T cells |
| IL-9 | Adaptive | T cells | IL-9R, | T cell | Growth and |
| | immunity | | CD132 | | proliferation |
| IL-10 | Anti- | T cells, B | CDw210 | B cells, | Inhibits |
| | inflammatory | cells, | | macrophages | cytokine |
| | | macrophages | | | production and |
| | | | | | mononuclear |
| | | | | | cell function |
| IL-11 | Pro- | BM stromal | IL-11Ra, | B cells | Differentiation, |
| | inflammatory | cells | CD130 | | induces acute |
| | | | | | phase proteins |
| IL-12 | Anti- | T cells, | CD212 | NK cells, | Activates NK |
| | inflammatory | macrophages, | | macrophages, | cells, |
| | | monocytes | | tumor cells | phagocyte cell |
| | | | | | activation, |
| | | | | | endotoxic |
| | | | | | shock, tumor |
| | | | | | cytotoxicity, |
| | | | | | cachexia |
| IL-17 | Pro- | Th17 cells | IL-17R | Monocytes, | Recruits |
| | inflammatory | | | neutrophils | monocytes and |
| | | | | | neutrophils to |
| | | | | | the site of |
| | | | | | infection. |
| | | | | | Activation of |
| | | | | | IL-17 in turn |
| | | | | | activate |
| | | | | | downstream of |
| | | | | | many |
| | | | | | cytokines and |
| | | | | | chemokine, |
| | | | | | such as IL-1, |
| | | | | | IL-6, IL-8, IL- |
| | | | | | 21, TNF-β, and |
| | | | | | MCP-1 |
| IL-18 | Pro- | Macrophages, | CD218a | Monocytes | Recruits |
| | inflammatory | dendritic | (IL-18Ra) | and T cells | monocytes and |
| | | cells, and | | | T |
| | | epithelial | | | lymphocytes. |
| | | cells | | | Synergist with |
| | | | | | IL-12 in the |
| | | | | | induction of |
| | | | | | IFN-γ |
| | | | | | production and |
| | | | | | inhibition of |
| | | | | | angiogenesis. |
| IL-22 | Anti- | Activated T- | IL-22R | Stromal and | Stimulation of |
| | inflammatory | cells and NK | | epithelial | cell survival, |
| | | cells | | cells | proliferation |
| IL-37 (1L- | Anti- | B-cells, NK | CD218a | | Believed to act |
| 1F7) | inflammatory | cells, and | (IL-18Ra) | | as a negative |
| | | monocytes | and | | regulator |
| | | | potentially | | inside the cell |
| | | | SIGGR | | where it |
| | | | | | interacts with |
| | | | | | SMAD3 that is |
| | | | | | activated |
| | | | | | downstream of |
| | | | | | TGFβ activity. |
| IL-38 (IL- | Anti- | B cells and | IL-1R1 | | Unknown |
| 1F10) | inflammatory | macrophages | | | |
| IFN-α | Pro- | Macrophages, | CD118 | Various | Anti-viral |
| | inflammatory | neutrophils, | (IFNAR1, | | |
| | | and some | IFNAR2) | | |
| | | somatic cells | | | |
| IFN-β | Pro- | Fibroblasts | CD118 | Various | Anti-viral, |
| | inflammatory | | (IFNAR1, | | anti- |
| | | | IFNAR2) | | proliferative |
| IFN-γ | Pro- | T Cells and | CDw119 | Various | Anti-viral, |
| | inflammatory | NK cells | (IFNGR1) | | macrophage |
| | | | | | activation, |
| | | | | | increases |
| | | | | | neutrophil and |
| | | | | | monocyte |
| | | | | | function, |
| | | | | | MHC-I and -II |
| | | | | | expression on |
| | | | | | cells |
| M-CSF | Adaptive | Fibroblasts, | CD115 | Stem cells | Monocyte |
| | immunity | endothelium | | | production and |
| | | | | | activation |
| TGF-β | Anti- | T cells and B | TGF-βR1, | Activated T | Inhibits T and |
| | inflammatory | cells | 2, 3 | and B cells | B cell |
| | | | | | proliferation, |
| | | | | | inhibits |
| | | | | | hematopoiesis, |
| | | | | | promote |
| | | | | | wound healing |
| TNF-α | Pro- | Macrophages | CD120a, b | Macrophages | Phagocyte cell |
| | inflammatory | | | | activation, |
| | | | | | endotoxic |
| | | | | | shock |
| TNF-β | Pro- | T Cells | CD120a, b | Phagocytes, | Chemotactic, |
| | inflammatory | | | tumor cells | phagocytosis, |
| | | | | | oncostatic, |
| | | | | | induces other |
| | | | | | cytokines |

Abbreviations:
IL; interleukin,
TNF; tumor necrosis factor,
IFN; interferon,
G-CSF; granulocyte colony stimulating factor,
GM-CSF; granulocyte macrophage colony stimulating factor,
M-CSF; macrophage colony stimulating factor,
TGF; transforming growth factor,
CD; cluster of differentiation;
CDw; cluster of differentiation designated by only one monoclonal antibody,
BM; bone marrow,
DC; dendritic cells

As mentioned, the parameters described herein can be detected using methods which are well known in the art. Following are some examples for detection at the protein level or mRNA level. The specific method will be selected according to the knowledge of the skilled artisan and the level of sensitivity required, for example the ability to differentiate between different species.

According to some embodiments, analysing the inflammation parameter is at the protein level.

According to a specific embodiment, analyzing expression of said iron-regulated gene is at the protein level.

Expression and/or activity level of proteins expressed in the cells of the cultures of some embodiments of the invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabeled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

Specific methods, e.g., aconitase activity assay, Western blotting, staining and more are provided in the Examples section including examples for specific primary antibodies which are species specific.

According to some embodiments, analysing the inflammation parameter is at the mRNA level.

According to a specific embodiment, said analyzing expression of said iron-regulated gene is at the mRNA level.

Thus, the expression level of the RNA in the cells of some embodiments of the invention can be determined using methods known in the arts.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, CA).

Specific methods, e.g., PCR, and more are provided in the Examples section including examples for specific primers which are species specific.

Other methods of assessing the parameter can be considered. Following are some examples:

According to some embodiments, the phenotype is determined by analysing cellular distribution of the inflammation parameter.

According to some embodiments, the phenotype is determined by analysing post translational modification (e.g., phosphorylation) of the inflammation parameter.

The model described herein can find various uses in assessing pro or anti-inflammatory agents.

For example, in the determination of safety or efficacy of agents. Following are some non-limiting examples:

Pharmaceuticals: Safety assessments are crucial for pharmaceutical drugs to ensure their efficacy and minimize potential risks to patients. This includes evaluating the safety of active ingredients, excipients, dosage forms, and potential interactions with other medications.

Food and beverages: Safety evaluations are conducted for food additives, preservatives, flavorings, and food processing chemicals. These assessments help ensure that substances used in the food industry do not pose significant health risks to consumers.

Cosmetics and personal care products: Substances used in cosmetics, skincare products, haircare products, and other personal care items undergo safety assessments. This includes evaluating the safety of ingredients and potential risks associated with their use on the skin, hair, or nails.

Industrial chemicals: Safety evaluations are conducted for various industrial chemicals used in manufacturing processes, such as solvents, detergents, adhesives, and coatings. These assessments focus on worker safety, environmental impact, and potential hazards during production, use, and disposal. Agricultural chemicals: Pesticides, herbicides, and fertilizers undergo safety evaluations to assess their potential impacts on human health, wildlife, and the environment. These assessments help ensure that agricultural chemicals are used responsibly and do not pose undue risks.

Consumer products: Safety assessments are conducted for a wide range of consumer products, including household cleaning agents, paints, toys, electronics, and furniture. These evaluations aim to identify and mitigate potential hazards to consumers during normal use.

Environmental substances: Safety evaluations are conducted for substances that may have an impact on the environment, such as pollutants, industrial emissions, waste products, or hazardous materials. These assessments help identify potential risks to ecosystems and human populations.

Alternatively, the model can be used to determine the efficacy of drugs as pro or anti inflammatory cytokines.

Hence contemplated herewith is the use of the model in drug screening.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

### Human Terminal Ileum Tissues

Paraffin embedded blocks of the terminal ileum segments used for this study were provided by the histology unit at RAMBAM hospital. Specimen were obtained from patients undergoing routine endoscopy or colonoscopy as part of their standard medical care. Each patient gave informed consent, and the study protocol was approved by the institutional committee. Study subjects were diagnosed with clinically active Crohn's disease (CD). The diagnosis of CD was based on standard clinical, radiological, endoscopic, and histologic criteria. Biopsies were obtained from both, inflamed and normal-appearing segments of mucosa from the same patient.

### Mouse Studies

Animal studies were carried out in accordance with the recommendations in the Guide for the Care and Use of Laboratory Animals of the pre-clinical research authority at the Technion, Haifa, Israel. The protocols were approved by the Technion Animal Ethics Committee, Haifa, Israel. IRP1 knockout mice on C57BL/6 background (IRP1^{-/-}) were described in EMBO journal 23, 386-395 (2004). TNF^{ΔARE/+} mice on C57BL/6 background (TNF^{ΔARE/+}) were described in Kontoyiannis et al., Immunity 10, 387-398 (1999). WT littermates from breeders of TNF^{ΔARE/+} with WT mice were used as controls in the experiments described here. TNF^{ΔARE/+} mice with targeted deletion of IRP1 were generated by crossing TNF^{ΔARE/+} mice with IRP1^{-/-}. IRP1^{-/-} littermates from the former breeders were used as controls in the experiments described here. All mice were housed in the specific pathogen-free animal facilities at the animal facility of the Technion, Haifa, Israel.

### Iron Overload Mouse Models

Fe-dextran (SIGMA) (90 mg/ml) was injected intra-peritoneally to 12-13 weeks old mice for five days as previously described [Weiss et al. PLoS One. 2018 Oct. 15; 13(10):e0204471. PMID: 30321179; PMCID: PMC6188744]. The mice were sacrificed three days after the last injection.

### DSS Model

C57BL/6J (WT) mice aged 6-8 weeks were treated with 4% DSS (36,000-50,000 Da, MP Biomedicals, Solon, OH, USA) in drinking water for 7 days. Body weight and blood in stool were monitored daily.

### Tissue Fixation

Terminal ileum was collected from 12-14 weeks old mice. Animals were euthanized and terminal ileum was dissected. Tissues were then first rinsed with PBS and fixed with 4% PFA solution (Electron Microscopy Sciences) for at least 16 hours, then dehydrated into 70% ethanol. Tissues were subsequently embedded in paraffin by the pathology unit in RAMBAM hospital. Blocks were then sectioned using a Shandon Finesse 325 Manual Rotary Microtom (Thermo). Sections were fixed onto Superfrost microscope slides. Sections were then deparaffinized with Xylene and rehydrated in graded EtOH.

### Immuno-Fluorescent Staining

For immune-fluorescent staining, paraffin embedded terminal ileum sections prepared as described, were stained with Affinity purified rabbit anti mouse liver ferritin [as described in Konijn et al., J Immunol Methods 54, 297-307 (1982)], and then probed with Alexa Fluor 488 conjugated anti-rabbit antibody (Invitrogen). Fluorescent signal was quantified using IMARIS software.

### Hematoxylin and Eosin (H&E) Staining

For H&E staining, paraffin embedded terminal ileum sections were deparaffinized with Xylene and rehydrated in 100% EtOH then in 95% EtOH. Staining was performed with Harris Hematoxylin solution (Sigma) and Eosin Y (Sigma).

### Immunohistochemistry Staining

For immunohistochemistry, paraffin tissues sections were prepared as described. The sections were then put into 3% H₂O₂ for 15 min and boiled in 10 mM citrate buffer for 10 min then blocked with 1% BSA for 1-2 h. Tissue sections were incubated with primary antibody against FtH (1:1000) and probed with secondary antibody (1:10000, diluted in 1% BSA) for 3 h at room temperature. The reaction was attenuated, dehydrated and sealed after 3,3-diaminobenzidin (DAB, ZSGB-BIO, Beijing, China) color rendering. Quantification of the brown-yellow density was performed with ImageJ

### Assessment of Inflammatory Bowel Disease (IBD)

H&E stained sections were blindly presented to a certified pathologist. Assessment of disease severity was based on standard histologic criteria.

### Perls' Prussian Blue Iron Staining

For Perls' Prussian blue staining, paraffin embedded terminal ileum sections prepared as described, were incubated with freshly prepared Perls' Prussian blue solution (2% hydrochloric acid mixed with 2% potassium ferrocyanide to reveal iron contents and distribution in the sections. Quantification was evaluated by morphometric means.

### Tissue Fractionation

Mice were sacrificed at 12-14 weeks of age. Animals were euthanized and the terminal ileum was dissected, opened and rinsed with cold deoxygenated PBS. First epithelial cells were isolated as follows, the tissue was cut into segments, and immersed in HBSS (Biological Industries) supplemented with 20% FBS, 1 mM DTT and 2 mM EDTA. The segments were then incubated in 37° C. for 30 min in shaking. The solution was then transferred through grey mesh laminate. Suspended cells were then pelleted by centrifugation at 212×g and washed twice with PBS.

Immune cells were isolated from the segments by immersing the remaining tissue in PBS with 0.492 mM CaCl₂) and 0.9 mM MgCl₂ supplemented with 20% FCS, collagenase 1 mg/ml (Sigma) and DNAse 0.1 mg/ml (Sigma). The Segments were then incubated in 37° C. for 30 min in shaking. The solution was then transferred through grey mesh laminate. Suspended cells were then pelleted by centrifugation at 212×g and washed twice with PBS. Cells were snap frozen in liquid nitrogen and stored in −80° C. for further use. Tissue fractionation was done in anaerobic environment. All solutions used were degassed.

### Cultivation of Cell Lines

Caco2 cells (ATCC CR2 2101) and Raw 264.7 cells (a kind gift of Prf. B. Levi, Technion) were cultured in DMEM (Sigma,) supplemented with 10% heat inactivated FCS (Biological Industries), L-glutamine (Biological Industries) and Pen.-Strep. (Biological Industries) at 37° C. and 5% CO₂. Both cell lines were tested for mycoplasma contamination.

### Primary Cultures of Bone Marrow Derived Macrophages (BMDM)

Total bone marrow harvested from tibia and femur of 8-12 month old mice was cultured for 6 days in a regular cell culture incubator, in DMEM medium containing 20% FCS, 30% CCL1 cell-conditioned medium (CCL1 cells as described in Fleit and Rabinovitch J. Cell Physiol. 1981 108(3):347-52) L-glutamine and Pen.-Strep. On day six cells were scraped on ice and re-plated in Dulbecco's PBS (Sigma) for 30 minutes. The PBS was then replaced by DMEM containing 10% FCS, Pen.-Strep. and glutamine.

### Inter-Species Co-Culture Assembly

Day before co-culture assembly Caco2 cells were cultivated in an oxygen modified incubator (6% O₂). Confluent co-cultures of epithelial cells and macrophages were obtained by seeding 2×10⁵ cells per cm² surface area. Cell culture dishes employed were 10 cm or 6 well. For Caco2-Raw 264.7 co-cultures, Caco2 cells were seeded, after adherence Raw264.7 cells were added accordingly. For Caco2-BMDM co-cultures, on the six day of bone marrow extraction the cells were plated as previously mentioned, and Caco2 cells were added to them accordingly. Co-cultures were cultivated in an oxygen modified incubator (6% O₂) at 37° C. and 5% CO₂. Co-culture ratios used were 1:1 or 2:1 or 1:2.

### S-Nitroso-N-Acetylpenicillamine (SNAP) Treatment

Day before SNAP (Sigma) treatment, Caco2 cells were cultured in working medium with MEM (Sigma) supplemented with 20% heat inactivated FCS (Biological Industries), ferric ammonium citrate (Sigma), non-essential amino acids (Gibco), sodium pyruvate (Gibco), L-glutamine (Biological Industries) and Pen.-strep. (Biological Industries) in an oxygen modified incubator (6% O₂) at 37° C. and 5% CO₂.

SNAP was added to the cells for 22 hours in two sequential doses 1 mM each. First dose was added for 6 hours, 20% of the medium was then collected and replaced with working medium containing 5 mM SNAP for additional incubation of 15 hr.

### Cell Lysate Preparation

Cells were lysed in degassed lysis buffer consisting of 10 mM HEPES pH 7.2, 3 mM MgCl₂, 40 mM KCl, 5% Glycerol, 0.2% Nonidet P-40, 5 mM DTT, 1 mM AEBSF, 10 g/ml Leupeptin and Complete™ EDTA free protease inhibitor cocktail (Roche Applied Science, Indiana), for 10 min. Nuclei and debris were removed by centrifugation. Preparation of cell lysates was done in anaerobic chamber. Protein concentrations were determined using the Bradford assay (BIO-RAD, Hercules, California)

### Western Blotting and Antibodies

Equal amounts of protein (40 μg/lane) were separated on SDS-PAGE and transferred to nitrocellulose membranes. The membrane was blocked with blocking solution and probed at 4° C. as described in Table 1. Secondary antibody was horse-radish peroxidase-conjugated IgG as described in Table 1. Blots were developed using enhanced chemiluminescence (ECL kit, Pierce, Illinois).

### RNA Mobility Shift Assays.

Gel retardation assays were performed as described [Haile et al. Mol Cell Biol. 1989 November; 9(11):5055-61. PMID: 2601708; PMCID: PMC363657]. Cell lysates were prepared as described above. Equal amounts of about 5 g of total protein were added to a final volume of 12.5 μl buffer containing 25 mM Tris HCl pH 7.5, 40 mM KCl with or without 2% 2-mercaptoethanol (ME), which activates IRP1 in vitro. The samples were incubated for 5 minutes at room temperature with 12.5 μl of a reaction cocktail containing 20% glycerol, 0.2 U/μl Super RNAsine (Ambion, Texas), 0.6 μg/μl yeast tRNA, 5 mM DTT and 20 nM 32P-labelled IRE from the human ferritin H chain gene in 25 mM Tris HCl pH 7.5, and 40 mM KCl. 20 μl of this reaction mixture were loaded onto a 10% acrylamide/TBE gel, that was run at 200 V for 2.25 hours, then the gel was fixed, dried and exposed for autoradiography.

### Aconitase Assay

Mitochondrial and cytosolic aconitase activities were assayed in parallel in cell lysates after electrophoretic separation as previously described [Tong et al. Cell metabolism 3, 199-210 (2006)]. Protein concentrations were determined using the Bradford assay (BIO-RAD, Hercules, California). Aconitase activity was detected chromogenically by incubating the gels in 100 mM Tris pH 8, 1 mM NADP, 2.5 mM cis-aconitic acid, 1.2 mM 3-(4,5-dimethylthiazol-2-yl)-2,5-diphenyl tetrazolium bromide, 0.3 mM phenazine methosulfate, 5 mM MgCl₂ and 5 Units/ml isocitrate dehydrogenase. All chemicals were purchased from Sigma Missouri.

### Iron Stain for Gels

Cell lysates were prepared as previously described. Tissues were first ground in liquid-N2 cooled mortars then lysed the same. An SDS-free sample buffer was added to lysate samples, followed by heating at 70° C. for 10 minutes and centrifugation at 10000×g for 5 minutes. Samples were resolved on a native-PAGE in non-reducing conditions. The gel was stained with Perl's Prussian Blue staining solution for 1 hour at RT, then rinsed until reaching a close to neutral pH and incubated in 100 mM Tris buffer (pH 7) for 5 minutes. Next, the ferric iron stain was enhanced by 3, 3′-Diaminutesobenzidine (DAB) enhancement solution (Arcos Organics) for ON at RT.

Labeling Cells with ⁵⁵Fe

Cells were given medium containing transferrin loaded with labeled iron. First labeled Ferric nitrilotriacetate (Fe-(NTA)₂) was prepared, by mixing 5 parts of 200M NTA (pH=3) in 0.9% NaCl, with 4 parts of 100 mM of ferric chloride solution (FeCl₃) in 0.1M HCl and one part of ⁵⁵Fe labeled ferric chloride solution (⁵⁵FeCl₃). The mixture was incubated for 30 minutes at RT. Next, 125 parts of 1 mM human Apotransferrin (Kamada) solution in HBS was mixed with 12.5 parts of NaHCO₃ and 10 parts of the labeled Ferric nitrilotriacetate (50 mM). The mixture was then added to the growth medium, 147.5 ul of labeled transferrin was added to 2 ml of growth medium. Cells were labeled with ⁵⁵Fe for ON. Then the medium was removed and cells were washed with PBS.

Inducing inflammation in ⁵⁵Fe labeled co-cultures: in order to induce inflammation in the co-cultures, LPS 200 ng/ml was added for indicated times. LPS-stimulated cells and control non-stimulated cells were harvested at each time point then snap frozen and kept at −80° C. for further use.

SNAP treatment in ⁵⁵Fe labeled Caco2 cells: cells were treated with SNAP as described before. After ON of SNAP incubation cells were harvested at each time point then snap frozen and kept at −80° C. for further use.

### Subcellular Fractionation

The cells were first suspended in lysis buffer consisting of 10 mM HEPES pH 7.9, 10 mM KCl, 1.5 mM MgCl₂, 0.34 M Sucrose, 10% Glycerol, 1 mM DTT, 1 mM AEBSF and Complete™ EDTA free protease inhibitor cocktail (Roche Applied Science, Indiana). 0.1% Triton was then added to the cell suspension for 5 minutes. Cells were centrifuged at 1500×g for 4 minutes. The supernatants were transferred into new tubes and kept as the crude cytoplasmic enriched fraction. The pellet was then washed with the first lysis buffer then lysed in a second lysis buffer consisting of 3 mM EDTA, 0.2 mM EGTA, 1 mM DTT, 1 mM AEBSF and Complete™ EDTA free protease inhibitor cocktail (Roche Applied Science, Indiana), for 10 minutes then centrifuged at 1700×g for 4 minutes. The supernatant was transferred into new tube and was kept as nucleoplasmic fraction. The pellet was first washed in the second lysis buffer then suspended in the first lysis buffer added to 1 mM CaCl₂) and 0.6 U of Micrococcal Nuclease (MNase) and incubated for 30 minutes at 37° C. Then centrifuged at 20,000×g for 10 minutes. The supernatant was transferred into new tube and was kept as chromatin bound fraction. The crude cytosolic enriched fraction was further centrifuged at 20,000×g for 10 minutes, the supernatant was transferred into new tube and was kept as cytosolic enriched fraction. All steps were performed in an anaerobic environment. All lysis buffers used were degassed.

### Metabolic Labeling and Immunoprecipitation

Cells were metabolically labeled for 1 hour, with S35 translabel, 1 mCi/5 ml medium, in DMEM without cysteine and methionine, 10% dialyzed FCS, 10 mM HEPES, pen./strep and glutamine. Immunoprecipitation was done with Protein-A sepharose beads (GE-Healthcare). Beads were prepared as per the manufacturer's instructions and conjugated with anti-FtH antibodies. Cells were lysed in lysis buffer consisting of 1% Triton X-100 (Bio-Lab), 20 mM Tris pH 8.0 (Sigma-Aldrich), 37 mM NaCl (Bio-Lab), and 10% Glycerol (Bio-Lab). One tablet of EDTA-free protease inhibitors cocktail (Roche). Lysates were first pre-cleared with un-coated protein-A Sepharose beads, then supernatants from the pre-cleared samples were transferred to fresh tubes containing slurry of protein-A Sepharose beads coated with anti-FtH antibodies. Samples were rotated for 3 hours at 4° C. and centrifuged at 800×g. The immunoprecipitated proteins were denatured with reducing sample buffer x1 and boiled at 95° C. for 5 minutes. Samples were then resolved on 14% SDS-PAGE. The gel was fixed, dried and exposed for autoradiography.

### Quantitative RT-PCR

RNA was extracted using the Trizol reagent (Invitrogen). Cells were harvested directly with Trizol, whereas tissues were first ground in liquid-N2 cooled mortars then incubated with Trizol following manufacturer's instructions. Isolated RNA samples were submitted to DNase treatment using DNase I recombinant, RNase-free kit (Roche). cDNA was prepared using cDNA Reverse Transcription Kit (Quanta biosciences), and the RT-PCRs were performed using SYBR master mix (Applied Biosystems). All primers used in the study were shown in Table 5 and in Table 6. The delta Ct method was used to calculate a relative expression of the target gene in comparison to topoisomerase 1 for epithelial cells and ubiquitin for macrophage genes.

### IL8 Elisa

IL8 concentration in the medium was evaluated with colorigenic IL8 Elisa kit (Sigma) as per the manufacturer's instructions.

### Griess Assay

Nitrite concentration in the medium was determined via Griess assay. Briefly the medium was collected and centrifuged at 1000×g. 50 ul of the supernatant was added to 50 ul of 1% sulfanilamide in 5% phosphoric acid. The mixture was incubated in the dark for 10 min. Next 50 ul of 0.1 N-1-naphthylethylenediamine dihydrochloride (NED) was added. The reaction product was measured with the ELISA spectrocolorimeter (Multiskan EX, Thermo Electron Corporation), at 540 nm. Serial dilution of NaNO₂ served to create a standard curve of nitrite.

### Serum Ferritin Assay

Serum ferritin was measured by colorigenic ELISA essentially as described for human ferritin [Konijn et al., J. Immunol. Methods 54, 297-307 (1982)] with modifications. Coating was done with goat anti Rat Liver Ferritin. The primary antibody used for murine ferritin detection was Rabbit anti mouse liver Ferritin. And the secondary conjugated antibody used was goat anti Rabbit IgG beta-galactosidase conjugated Human absorbed (Southern Biotech). Reaction product from the reaction substrate, chlorophenol red β-D-galactopyranoside (CPRG), was measured with the ELISA spectrocolorimeter (Multiskan EX, Thermo Electron Corporation), at 595 nm.

TABLE 1
Blocking and 1^{st} antibody conditions for in vivo Western blot analysis

| | Gel | | | | | | | | |
|---|---|---|---|---|---|---|---|---|---|
| Antigen | (%) | Blocking | Time | Temp. | 1^{st} Antibody | Dilution | Solution | Temp. | Time |
| Actin | 12% | 5% BSA | 1 hr | RT | Santacruz, | 1:2500 | 5% BSA | RT | 1 hr |
| | | | | | sc-1616 | | | | |
| TfR1 | 12% | 5% BSA | 1 hr | RT | Abcam, cat# | 1:2000 | 5% BSA | 4° C. | ON |
| | | | | | ab84036 | | | | |
| IRP2 | 12% | 5% milk | 1 hr | RT | a kind gift | 1:2000 | 5% milk | 4° C. | ON |
| | | | | | of Dr. | | | | |
| | | | | | Tracey | | | | |
| | | | | | Rouault | | | | |

TABLE 2
2^{nd} antibody conditions for in vivo Western blot analysis

| Antigen | 2^{nd} Antibody | Dilution | Solution | Temp. | Time |
|---|---|---|---|---|---|
| TfR1 | Horseradish-peroxidase | 1:25,000 | TBST | RT | 1 hr |
| | conjugated goat anti- | | | | |
| | rabbit antibody (Abcam | | | | |
| | ab97200) | | | | |
| Actin | Horseradish-peroxidase | 1:10,000 | TBST | RT | 1 hr |
| | conjugated anti goat | | | | |
| | (Sigma A5420) | | | | |

TABLE 3
Blocking and 1^{st} antibody conditions for in vitro Western blot analysis

| | Lower | Blocking | | | 1^{st} | | | | |
|---|---|---|---|---|---|---|---|---|---|
| Antigen | gel (%) | solution | Time | Temp. | Antibody | Dilution | Solution | Temp. | Time |
| Actin | 12% | 5% BSA | 1 hr | RT | sc-1616 | 1:10,000 | 5% BSA | RT | 1 hr |
| TfR1 | 6% | 5% BSA | 1 hr | RT | Invitrogen | 1:500 | TBST | 4° C. | ON |
| | | | | | 136800 | | | | |
| IRP2 | 12% | 5% milk | 1 hr | RT | Klausner | 1:200 | 5% milk | 4° C. | ON |
| | | | | | et al., | | | | |
| | | | | | EMBO J. | | | | |
| | | | | | 1995 Nov. | | | | |
| | | | | | 1; 14(21): | | | | |
| | | | | | 5350-7. | | | | |
| | | | | | PMID: | | | | |
| | | | | | 7489724; | | | | |
| | | | | | PMCID: | | | | |
| | | | | | PMC394644. | | | | |
| Ft-H | 12% | 2% milk | 1 hr | RT | Cell | 1:1000 | 2% milk | 4° C. | ON |
| | | | | | signaling | | | | |
| | | | | | D1D14 | | | | |
| Ft-L | 12% | 1% milk | 1 hr | RT | Ab 69090 | 1:1000 | 1% milk | 4° C. | ON |
| Hif 2 α | 10% | 5% milk | 1 hr | RT | NB100- | 1:500 | 5% milk | 4° C. | ON |
| | | | | | 122b | | | | |
| ERK 1/2 | 12% | 5% BSA | 1 hr | RT | sc-514302 | 1:1500 | 1% BSA | 4° C. | ON |
| p-ERK | 12% | 5% BSA | 1 hr | RT | sc-7383 | 1:1500 | 1% BSA | 4° C. | ON |
| 1/2 | | | | | | | | | |
| p-Nfkb | 12% | 5% milk | 1 hr | RT | sc-136548 | 1:500 | 1% BSA | 4° C. | ON |
| 65 | | | | | | | | | |
| P38 | 12% | 5% milk | 1 hr | RT | sc-81621 | 1:500 | 1% BSA | 4° C. | ON |
| p-p38 | 12% | 5% milk | 1 hr | RT | sc-7973 | 1:500 | 1% BSA | 4° C. | ON |

TABLE 4
2^{nd} antibody conditions for in vitro Western blot analysis

| | | | Solu- | | |
|---|---|---|---|---|---|
| Antigen | 2^{nd} Antibody | Dilution | tion | Temp. | Time |
| IRP2 | Horseradish-peroxidase | 1:25,000 | TBST | RT | 1 hr |
| Ft-H | conjugated goat anti | | | | |
| Ft-L | rabbit(Abcam) | | | | |
| TfR1 | Horseradish-peroxidase | 1:10,000 | TBST | RT | 1 hr |
| ERK1/2 | conjugated anti mouse | | | | |
| p- | | | | | |
| ERK1/2 | | | | | |
| P-NfKB | | | | | |
| p65 | | | | | |
| p38 | | | | | |
| p-p38 | | | | | |
| Hif2α | Horseradish-peroxidase | 1:25,000 | 5% | RT | 1 hr |
| | conjugated goat anti | | milk | | |
| | rabbit(Abcam) | | | | |
| Actin | Horseradish-peroxidase | 1:10,000 | TBST | RT | 1 hr |
| | conjugated anti goat | | | | |
| | (Sigma A5420) | | | | |

TABLE 5
Sequence of murine primers used in quantitative PCR analysis

| Primer name | Primer sequence |
|---|---|
| mUb Forward | CCA GTG TTA CCA CCA AGA AG/1 |
| mUb Reverse | ACC CAA GAA CAA GCA CAA GG/2 |
| mTNF-α Forward | CAA ATT CGA GTG ACA AGC CTG/3 |
| mTNF-α Reverse | GAG ATC CAT GCC GTT GGC/4 |
| m Icam1 Forward | CAA TTT CTC ATG CCG CAC AG/5 |
| m Icam 1 Reverse | AGC TGG AAG ATC GAA AGT CCG/6 |
| m IL 1β Forward | GCC CAT CCT CTG TGA CTC AT/7 |
| m IL 1β Reverse | AGG CCA CAG GTA TTT TGT CG/8 |
| m IL 6 Forward | AGC CAG AGT CCT TCA GAG AGA T/9 |
| m IL 6 Reverse | TGT TAG GAG AGC ATT GAG AAT TGG/10 |
| m TfR1 Forward | GTG GAG TAT CAC TTC CTG TCG/11 |
| m TfR1 Reverse | CCC CAG AAG ATA TGT CGG AAA GG/12 |
| m DMT1 Forward | CAT CCT GGT CCT GAT CGT CTGC/13 |
| m DMT1 Reverse | CCA CAT AGA GTG CCA CAT GCC CTAG/14 |
| m Fpn 1 + IRE Forward | AGG CTT TGG CTT CCA ACT TCA GC/15 |
| m Fpn 1 + IRE Reverse | AAG CAC AAC AGC CTT ATG CCG AAA G/16 |
| m Ft H Forward | AAG TGC GCC AGA ACT ACC AC/17 |
| m Ft H Reverse | TTC AGA GCC ACA TCA TCT CG/18 |
| m Ft L Forward | AAT CAG GCC CTC TTG GAT CT/19 |
| m Ft L Reverse | GGT TGC CCA TCT TCT TGA TG/20 |
| m NCOA4 Forward | AAG AAA GTG GGA AGC CTC AG/21 |
| m NCOA4 Reverse | AGA TCA CAA ACT GCT GGG AG/22 |
| m Fxn Forward | TAC ATG TCA CAG TCA ACG CC/23 |
| m Fxn Reverse | CTC GTC TAG AGA GCT TGG GT/24 |
| m Hsc20 Forward | AAT GAA AGA CTC GCA GAC GC/25 |
| m Hsc20 Reverse | AAG TCA CCT TGT TCA AAA GCG/26 |
| m Iscu Forward | GAT TGT GGA CGC CAG ATT CA/27 |
| m Iscu Reverse | GGC TTC CTC CAC CGT TTT C/28 |
| m sd11 Forward | ATA ATC CGC AGA CAG GTC CA/29 |
| m sd11 Reverse | TTC CTT GTG CTG GCT TCC TA/30 |

TABLE 6
Sequence of human primers used in quantitative PCR analysis

| Primer name | Primer sequence |
|---|---|
| h Top1 Forward | GGT GAG AAG GAC TGG CAG AAA T/31 |
| h Top1 Reverse | CTT GTC GAT GAA GTA CAG GGC TA/32 |
| h IL8 Forward | ATGAGTTCCAAGCTGGCCGTGGCT/33 |
| h IL8 Reverse | TCTCAGCCCTCTTCAAAAACTTCT/34 |
| h cxcl10 Forward | CTG CCA TTC TGA TTT GCT GC/35 |
| h cxcl10 Reverse | CGT ACA GTT CTA GAG AGA GG/36 |
| h Icam1 Forward | TTG GGC ATA GAG ACC CCG TT/37 |
| h Icam1 Reverse | GCA CAT TGC TCA GTT CAT ACA CC/38 |
| h TfR1 Forward | TGG AGA CTT TGG ATC GGT TGG TG/39 |
| h TfR1 Reverse | CAG TGG CTG GCA GAA ACC TTG/40 |
| h DMT1 Forward | CAC CAT GAC AGG AAC CTA TTC TGG C/41 |
| h DMT1 Reverse | AAC CAC TCG GGC AAA GCG TGA C/42 |
| h Fpn 1-IRE Forward | AAA CGA AGT CAA CCA AGG CTA CAG TC/43 |
| h Fpn 1-IRE Reverse | ACA GGA GTG CAA GGA ACT GGA GAT AG/44 |
| h Ft H Forward | AAA CGC GAA TTC ACC ATG ACG ACC GCG/45 |
| h Ft H Reverse | ACC GCG GCC GCT TAG CTT TCA TTA TCA/46 |
| h Ft L Forward | GAA CCA GGC CCT TTT GGA TC/47 |
| h Ft L Reverse | CCA GGA AGT CAC AGA GAT GGG/48 |
| h NCOA4 Forward | CAA ATT CCT GAG CAC TTG ATG G/49 |
| h NCOA4 Reverse | AAA GGG ACA GCT ACA ATA CCG/50 |
| h Fxn Forward | ACT AGC AGA GGA AAC GCT GG/51 |
| h Fxn Reverse | GGA ATA GGC CAA GGA AGA CA/52 |
| h Nfs1 Forward | ACT CCC GGA CAC ATG CTT ATG/53 |
| h Nfs1 Reverse | GCT GAC GAG CAC GTT CCAT/54 |
| h Iop2 Forward | GGC GCT GAC TGC GTG TTA A/55 |
| h Iop2 Reverse | AGG TCA CCT TGC TCC ATT ATT TG/56 |
| h Isd Forward | AAA GCC AAG AGA GAC CTT GGA/57 |
| h Isd Reverse | CTA GGT CCT GGG CAT GTC TC/58 |

### Example 1

### a Modified Iron Homeostasis is Observed in the Inflamed Lesions of IBD Patients and Mouse Models.

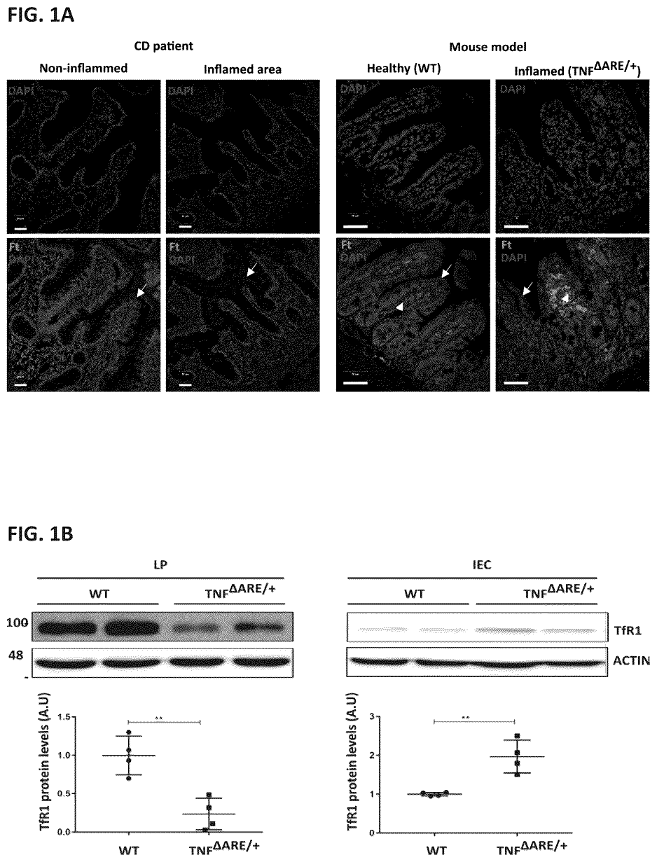

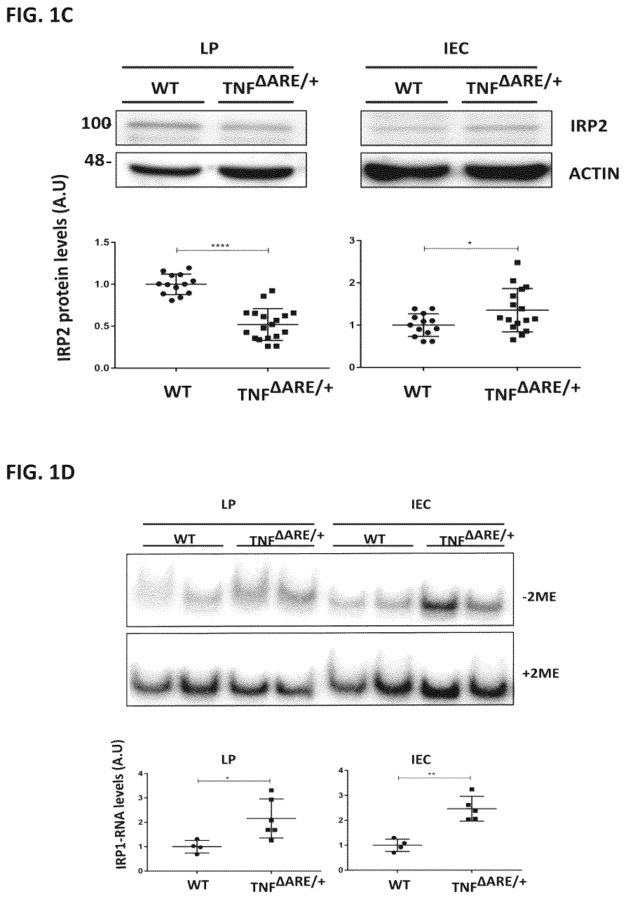

To evaluate iron homeostasis in the inflamed lesion of CD patients, the present inventors performed immune-fluorescent staining (IF) of the cellular iron-storage protein ferritin (a sensitive indicator for the iron status of cells,¹² in intestinal biopsies. Ferritin intensity was significantly reduced in intestinal epithelial cells (IECs) of the inflamed sections, compared to ferritin levels in IECs in non-inflamed areas from the same patients (FIG. 1A and not shows). The tumor necrosis factor alpha overexpressing mice (TNF^{ΔARE/+}), develop a spontaneous inflammation in the terminal ileum at the age of 12-14 weeks and rheumatoid arthritis later in life.¹³ In TNF^{ΔARE/+} mice, ferritin is accumulated in the immune cells, and similar to the observation in human biopsies, was about 20% decreased in IECs of the inflamed terminal ileum sections (FIG. 1A and not shown). Similar results were obtained in the DSS mouse model for colitis,¹⁴ where ferritin levels were significantly reduced in IECs of DSS treated mice compared to controls not shown). These results suggested that the inflammation causes a specific pattern of iron distribution, involving iron accumulation in the immune cells and iron deficiency in the IECs. To test if other iron related proteins respond similarly to this inflammatory iron-pattern, the present inventors prepared an IEC, and immune cell enriched (lamina propria-LP) fraction from the terminal ileum of the TNF^{ΔARE/+} mice. As ferritin, also Transferrin Receptor (TfR)1 is regulated post-transcriptionally by the IRP-system, yet in response to the different location of its IREs on the transcript, its cellular levels increase in iron deficiency and decrease in iron overload.^{15,16} Consistent with the inflammatory iron pattern, IEC-TfR1 was increased and LP-TfR1 was reduced, compared to the wild type (WT) fractions (FIG. 1B), supporting a modified iron homeostasis in these two cell-fractions. The present inventors then evaluated upstream cellular iron regulation and tested IRP2, which similarly to TfR1, is elevated in iron deprivation.^{17,18} Responsive to the intracellular iron state, IRP2 levels were increased in the inflamed IECs and decreased in the inflamed LP (FIG. 1C). However, IRP1-IRE-binding activity (normalized to the 2-Mercaptoethanol loading control), was significantly elevated in both fractions of the inflamed ileum compared to WT, regardless of the intracellular iron state (FIG. 1D).

### Example 2

### An Interspecies Co-Culture Model Mimics the Iron-Independent Inflammatory Activation of IRP1

The present inventor observed that inflamed epithelial cells take on an iron deprived phenotype, while inflamed immune cells take on an iron loaded phenotype, yet IRP1-RNA-binding activity was elevated in both cell-types. Therefore, they looked for an in vitro model, that enables to differentiate easily between the two cell-types and study the molecular mechanism underlying this intestinal inflammatory iron-pattern and its contribution to the course of the inflammation. Several co-culture models that successfully mimic different aspects of inflammation,¹⁹⁻²¹ did not mimic the inflammatory iron-pattern. The present results demonstrated that only cultures with direct cell to cell contact between IECs and macrophages fully mimicked the iron-pattern observed in vivo. Since it was difficult to differentiate between the IECs and the macrophages, the present inventors developed in vitro cellular interspecies co-cultures of the human epithelial enterocyte-like Caco2 cells, with either murine primary bone-marrow derived macrophages (BMDM) or the murine macrophage-like cell line Raw 264.7.²² LPS was used to elicit inflammation, which was validated, testing activation of key markers for signaling pathways involved in inflammation. Indeed, they found that the MAP-kinase pathways and NFkB p65, were activated by LPS (FIG. 6A). Also, the downstream response of proinflammatory cytokines including Il8 and ICAM1 in epithelial cells (FIG. 6B) and TNFα, ICAM1, Il1b and 116 in macrophages (FIG. 6C) were all activated in both co-cultures and dynamics of the LPS-triggered inflammatory response was nearly identical to published in vivo and in vitro models.²³⁻²⁵

Moreover, due to the slight differences in the molecular weight and consequently of the migration-rate of some human and mouse proteins (FIG. 6A) and the primer specificity to human or mouse genes (FIGS. 6B and C) the cell-types could be evaluated separately.

It was confirmed that inflammation was indeed induced in both cell-types. Macrophages alone responded to LPS in a similar manner as macrophages in co-cultures, yet the epithelial cells alone did not elicit an inflammatory response to LPS (FIG. 6D). In contrast, Caco2 cells alone, exposed to the nitric oxide donor S-Nitro-N-Acetyl-D,L-Penicillamine (SNAP) showed an inflammatory response (FIG. 6E).

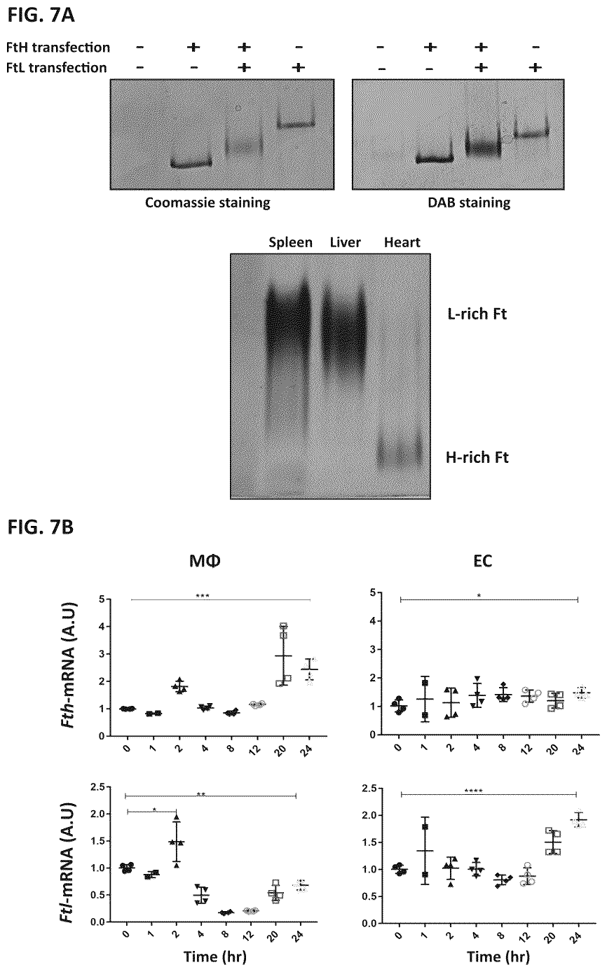

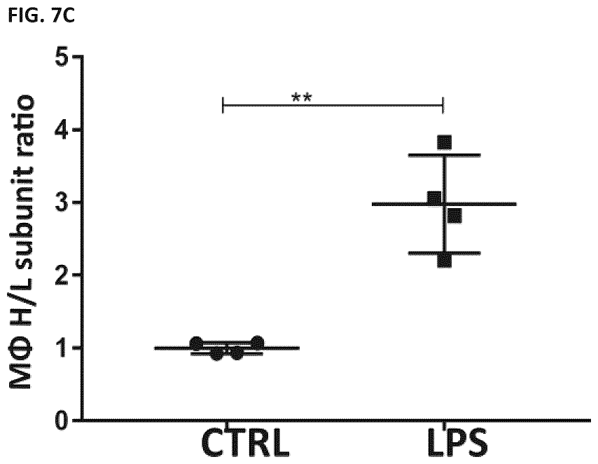

Further, in SNAP induced Caco2 cells, intracellular iron was reduced and extracellular iron increased (FIG. 7F) suggesting that one of the factors contributing to the inflammatory iron-pattern is nitric oxide.

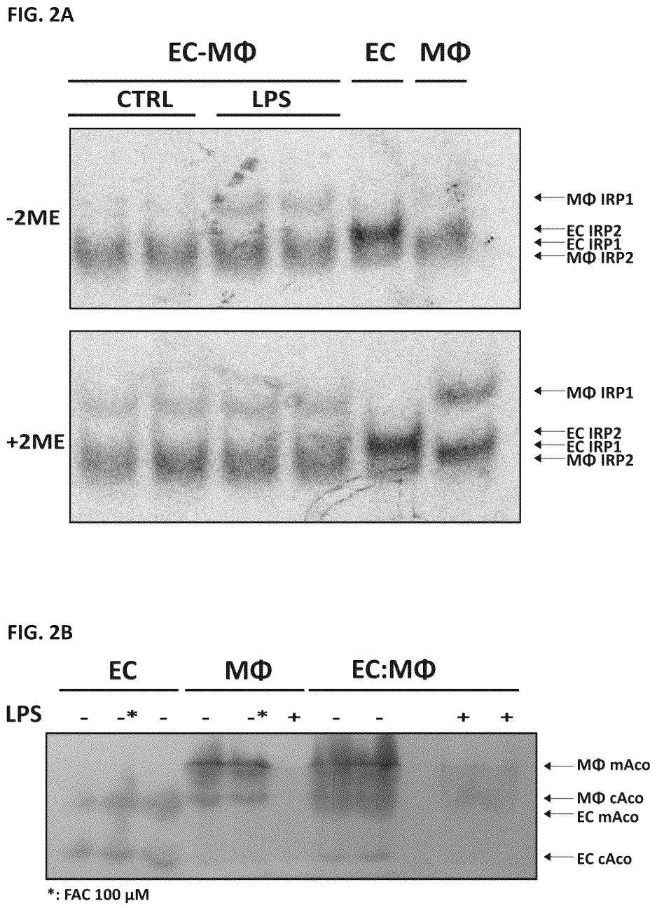

To test if LPS-induction of inflammation in the co-cultures will mimic the in vivo observations of modified iron homeostasis in the two cell-types, and iron-independent activation of IRP1, the present inventors evaluated the RNA binding activity of IRP1 via electromobility shift assay (EMSA) (FIG. 2A) RNA binding activity of macrophage IRP1 was significantly increased after LPS stimulation, similar to the observation in the TNF^{ΔARE/+} mice (FIG. 1D), yet epithelial IRP1 could not be appreciated as it was not separated from epithelial and macrophage IRP2 on the gel. Therefore, the present inventors evaluated IRP1 aconitase activity by the in-gel aconitase-assay, as reduced cAco aconitase activity correlates with increased IRP1-RNA-binding activity,²⁶ and optimized the gel-conditions to differentially examine human- and mouse-cAco-aconitase activity from epithelial cells and macrophages respectively (FIG. 2B). cAco activity was significantly decreased in response to LPS treatment, indicating an increase in the RNA-binding activity of IRP1 in both cell-types. Moreover, LPS-treatment also led to a significant decrease in mitochondrial aconitase (mAco) activity in both cell-types, supporting a metabolic shift to glycolysis as previously reported.^{27,28}

### Example 3

### Analysis of Inflammatory Ferritin-Metabolism, Using the Interspecies Co-Culture, Shows a Strong Increase in Macrophage Ferritin and a Shift in its Subunit Composition

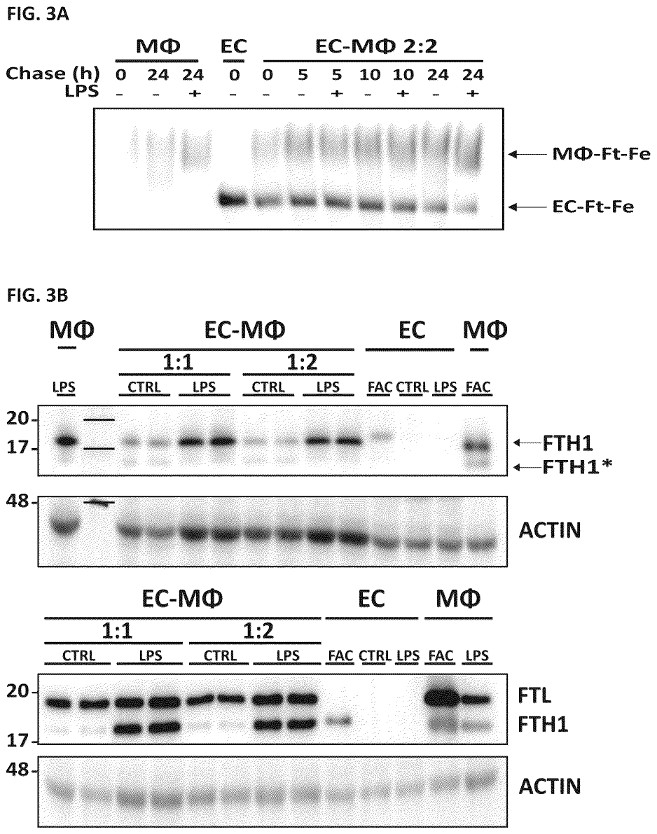

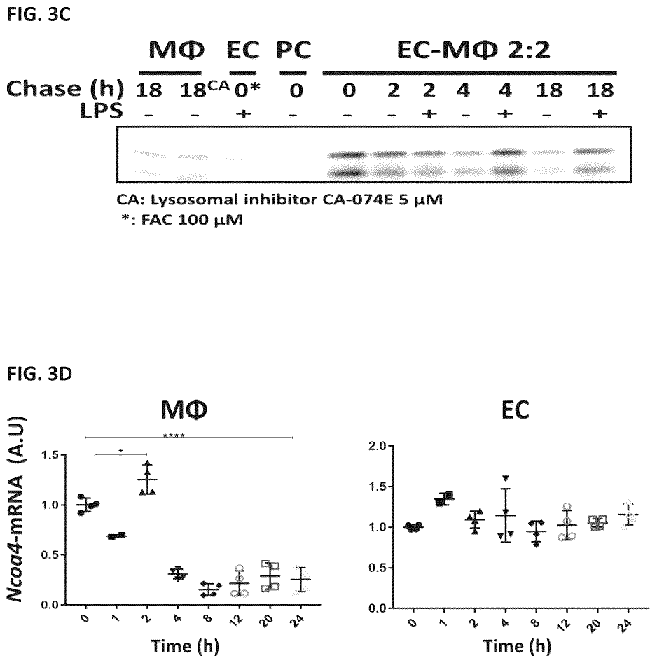

Reduction of IRP1 protein and activity was previously reported not to affect iron homeostasis in superoxide dismutase 1 knock-out mice,²⁹ yet the present inventors decided to test the effect of the increased IRP1 activity on the iron status and the IRP-regulated proteins in the two cell-types of the co-culture. First, they compared changes in ferritin-iron between LPS-stimulated and non-stimulated co-cultures by pulse-chase with transferrin-⁵⁵Fe. Twenty-four hours after LPS-stimulation, epithelial ferritin-iron was reduced and macrophage ferritin-iron was elevated compared to unstimulated cells at the same time-point (FIG. 3A). This suggested, that similarly to the observation in vivo, epithelial cells of the co-cultures take on an iron-deficiency-, and macrophages an iron-loaded-phenotype. The increased ferritin-iron in inflamed macrophages suggested that also ferritin-protein is elevated in these macrophages. Ferritin is a 24-subunit multimer, composed of two kinds of subunits, H-subunit (FTH1) and L-subunit (FTL).¹² The ratio of the subunits differs in various cell-types and pathophysiological conditions, due to transcriptional differences.³⁰ Yet, translational regulation is important in ferritin as transcripts of both subunits contain a 5′ IRE and translation is inhibited by active IRPs (usually a phenotype of iron-deficiency). Therefore, the present inventors wondered if the increase in ferritin-iron in macrophages was also accompanied by an increase in ferritin-protein, despite the translational inhibition by increased IRP1-IRE-binding. Indeed, a strong increase in macrophage ferritin-protein, especially of the FTH1 subunit was detected (FIG. 3B). In order to test if the increase observed in macrophage ferritin is due to a decrease in ferritin degradation, pulse-chase experiments of the co-cultures, metabolically labeled with ³⁵S just before stimulation with LPS were performed. Macrophage ferritin demonstrated a slower degradation of both subunits in LPS treated co-cultures compared to controls at the same time point (FIG. 3C). The observed decrease in ferritin degradation was further supported by the decrease of the transcripts of the ferritinophagy mediating NCOA4 in the inflamed BMDM (FIG. 3D). It was found that after 24 hours of LPS stimulation, macrophage-ferritin migrated slightly faster in the native gel (FIG. 3A), compared to its non-treated control, suggesting a shift in subunit composition of ferritin towards H-rich heteropolymers (FIG. 7A). Indeed, the transcript levels of FTH were strongly increased after LPS stimulation whereas transcript levels of FTL hardly changed (FIG. 7B). A similar increase of FTH1 was reported for monocytes exposed to inflammatory conditions.³¹ In agreement, the FTH:FTL protein ratio shifted from approximately 1:1 in the control macrophages to around 3:1 after LPS stimulation (FIG. 7C). However, in epithelial cells, FTH1 transcript levels increase only slightly, while FTL transcripts increase strongly upon LPS stimulation (FIG. 7B). Although epithelial ferritin-protein is very low, and mostly below detection, epithelial ferritin-iron was detected and decreased after 24 hours of LPS stimulation (FIG. 7A). Taken together, the complex dynamics of ferritin synthesis and degradation lead to an overall increase in macrophage ferritin, and decrease in epithelial ferritin iron, in agreement with the observations in the inflamed lesion of the TNF^{ΔARE/+} mice and the human IBD biopsies (FIG. 1A).

### Example 4

### In the Interspecies Co-Culture Model, TfR1 and IRP2 Mimic the Inflammatory Iron-Pattern

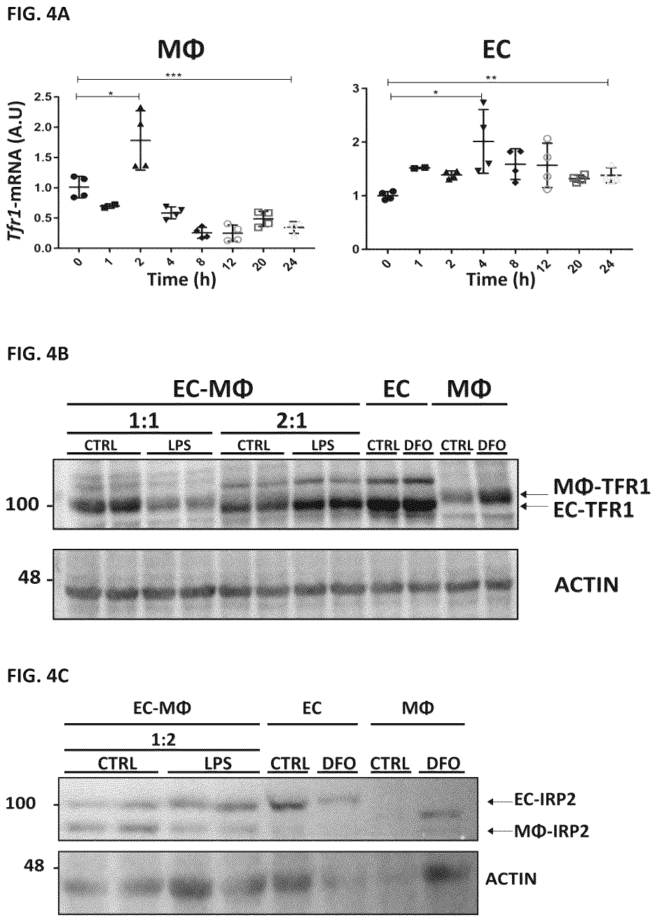

To further corroborate the inflammatory iron-pattern in the epithelial-macrophage co-culture, changes in mRNA levels following LPS induction, were evaluated for the iron importer TfR1 (FIG. 4A). Consistent with the iron accumulation in the macrophages and the iron decrease in the IECs of the TNF^{ΔARE} mice, TfR1 mRNA levels decreased 24 hours after LPS exposure in the macrophages and increased in the Caco2 cells. The TfR1 decrease in macrophages was preceded by an increase in TfR1 transcripts, two hours after LPS stimulation supporting an early inflammation mediated increase in iron import that leads to the iron accumulation in the macrophages (FIG. 4A). Protein levels of TfR1 could be differentially analyzed for epithelium and macrophages, due to the slight differences in the migration of human and mouse TfR1 in SDS-PAGE. Twenty-four hours after LPS-stimulation, TfR1 protein was increased in epithelium and decreased in macrophages according to the differential iron status of the two cell types (FIG. 4B). In addition, IRP2 responded to the inflammatory iron-pattern as expected and IRP2 levels were decreased in macrophages and slightly increased in epithelium (FIG. 4C) consistent with observations in the TNF^{ΔARE/+} mice (FIG. 1C).

### Example 5

### An Increase in the Hypoxia Inducible Factor (HIF)2α and a Decrease of Iron-Sulfur Cluster Assembly Proteins Support the Inflammatory Activation of IRP1-IRE Binding Activity

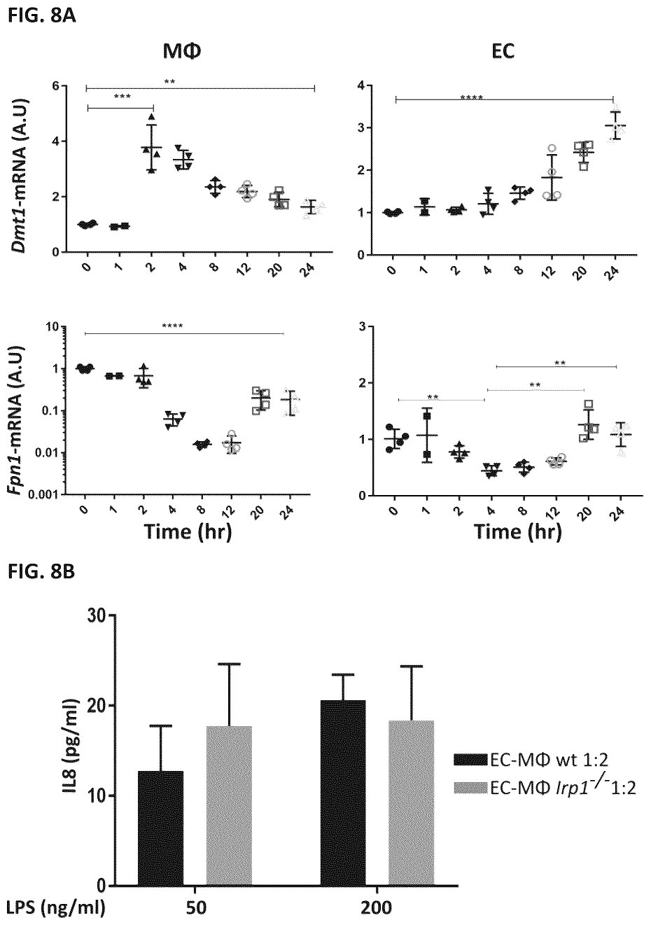

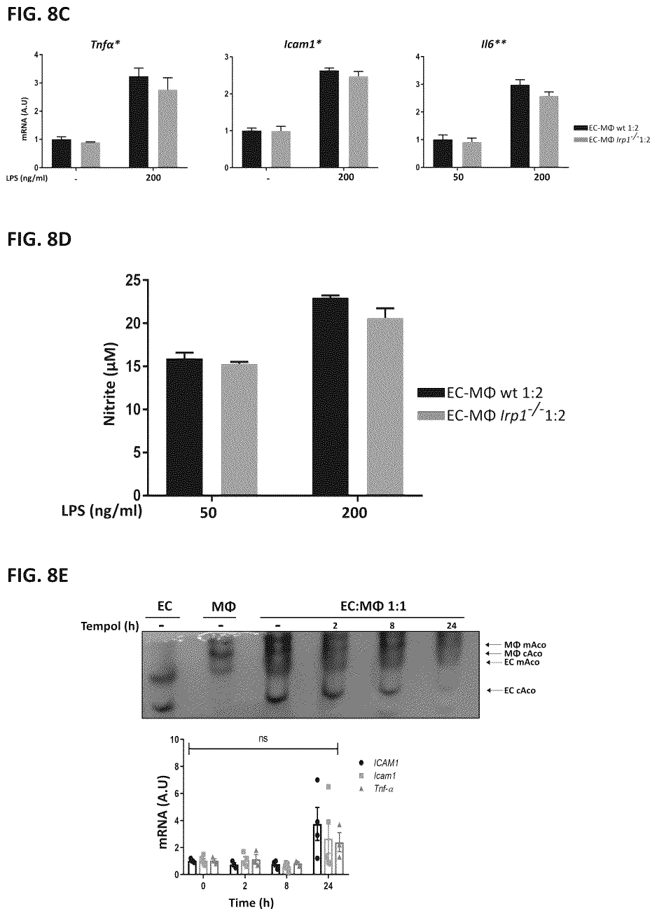

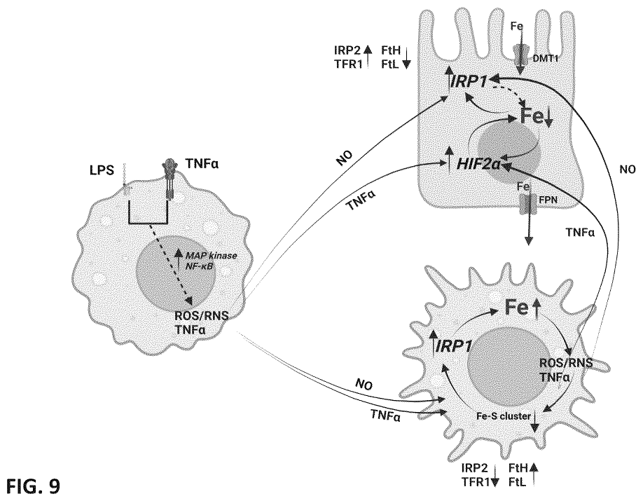

The present data suggested, that IRP1 is undergoing an inflammation-mediated and iron-independent activation in both cell-types and that this activation is supporting the development of a new iron-homeostasis of inflammation. To further elucidate the mechanism leading to the increased IRP1-IRE-binding activity in each cell-type, and based on the finding that Fe—S cluster assembly proteins Nfs1 and IscU mRNA and protein levels are decreased in BMDM in response to inflammation,³² the present inventors tested several genes involved in Fe—S cluster assembly. In macrophages, the transcription levels of all four iron-sulfur clusters biogenesis proteins tested, were significantly decreased, supporting the decrease in aconitase activities in mAco and cAco. Yet, in epithelial cells, transcripts of iron-sulfur cluster biogenesis proteins were not reduced (FIG. 5A). Nevertheless, epithelial aconitase activities were strongly decreased, suggesting an alternative mechanism for the loss of the Fe—S clusters in these proteins and the activation of IRP1 RNA binding activity. As the epithelial transcription regulator HIF2α is increased in IBD,³³ and HIF2α is a major regulator of iron uptake through epithelial cells,³⁴ the present inventors tested cytosolic and nuclear HIF2α and found them significantly increased in response to inflammation (FIG. 5B). In line with the HIF2α activation, both, the apical iron importer DMT1 and the basolateral iron exporter FPN increased gradually 4 hours after LPS stimulation (FIG. 8A) promoting both apical Fe-influx and basolateral Fe-efflux of the epithelial cells which further supports the epithelial iron-deficiency and consequently an increase in IRP1-RNA binding activity.

### Example 6

### Increased (Epithelial) IRP1 Activity Mediates the Inflammatory Iron Homeostasis and Contributes to the Course of Inflammation

Since IRP1 RNA binding activity was activated in macrophages regardless of their iron loaded phenotype, we asked if the absence of IRP1 in macrophages alone, will affect the inflammatory iron-pattern as seen above. LPS stimulated co-cultures of epithelial cells with IRP1^{-/-} macrophages responded similarly to the induction of inflammation as the cocultures with WT-macrophages. Specifically, FTH1 was significantly increased in macrophages of both inflamed co-cultures (Caco2 cells with either WT or IRP1−/− macrophages) (FIG. 5C). Also, IRP2 was significantly decreased in macrophages of both co-cultures, and epithelial IRP2 did not change in response to the LPS stimulation, regardless of the IRP1 genotype (not shown). Further, aconitase activity was significantly decreased in both co-cultures (not shown).

In addition, the inflammatory response of the co-culture with IRP1−/− macrophages was reminiscent of the co-cultures with WT macrophages in all parameters tested, including secreted epithelial cytokine IL8 and transcript levels of the macrophage inflammation markers TNFα, ICAM1 and IL6 (FIGS. 8B and C).

Also, the levels of the macrophage inflammatory mediator nitric oxide in the medium of both co-cultures were similarly increased in response to the LPS-stimulation (FIG. 8D). Altogether, these results suggested that the noncanonical activation of macrophage IRP1 alone, doesn't lead to the generation of the inflammatory iron-pattern. As epithelial cells have been shown to take on a driving force in the pathophysiology of IBD,^{35,36} and epithelial iron deficiency was associated with an inflammatory response³⁷ we tested if activation of epithelial IRP1 alone can drive an inflammatory response. To this end we activated IRP1 in the co-culture for indicated times with tempol.³⁸ Incubation with tempol alone did not elicit any inflammatory response in the co-cultures (FIG. 8E).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited in the Document

1 Kaplan, G. G. & Windsor, J. W. The four epidemiological stages in the global evolution of inflammatory bowel disease. Nature reviews. Gastroenterology & hepatology 18, 56-66, doi:10.1038/s41575-020-00360-x (2021).
2 Lih-Brody, L. et al. Increased oxidative stress and decreased antioxidant defenses in mucosa of inflammatory bowel disease. Digestive diseases and sciences 41, 2078-2086 (1996).
3 Yu, Y. & Richardson, D. R. Cellular iron depletion stimulates the JNK and p38 MAPK signaling transduction pathways, dissociation of ASK1-thioredoxin, and activation of ASK1. The Journal of biological chemistry 286, 15413-15427, doi:10.1074/jbc.M111.225946 (2011).
4 Steinbicker, A. U. & Muckenthaler, M. U. Out of balance—systemic iron homeostasis in iron-related disorders. Nutrients 5, 3034-3061, doi:10.3390/nu5083034 (2013).
5 Kroner, A. et al. TNF and increased intracellular iron alter macrophage polarization to a detrimental M1 phenotype in the injured spinal cord. Neuron 83, 1098-1116, doi:10.1016/j.neuron.2014.07.027 (2014).
6 Wilkinson, N. & Pantopoulos, K. The IRP/IRE system in vivo: insights from mouse models. Frontiers in pharmacology 5, 176, doi:10.3389/fphar.2014.00176 (2014).
7 Hanson, E. S. & Leibold, E. A. Regulation of the iron regulatory proteins by reactive nitrogen and oxygen species. Gene Expr 7, 367-376 (1999).
8 Koskenkorva-Frank, T. S., Weiss, G., Koppenol, W. H. & Burckhardt, S. The complex interplay of iron metabolism, reactive oxygen species, and reactive nitrogen species: insights into the potential of various iron therapies to induce oxidative and nitrosative stress. Free radical biology & medicine 65, 1174-1194, doi:10.1016/j.freeradbiomed.2013.09.001 (2013).
9 Kühn, L. C. Iron regulatory proteins and their role in controlling iron metabolism. Metallomics 7, 232-243, doi:10.1039/c4mt00164h (2015).
10 Meyron-Holtz, E. G. et al. Genetic ablations of iron regulatory proteins 1 and 2 reveal why iron regulatory protein 2 dominates iron homeostasis. The EMBO journal 23, 386-395 (2004).
11 Meyron-Holtz, E. G., Ghosh, M. C. & Rouault, T. A. Mammalian tissue oxygen levels modulate iron-regulatory protein activities in vivo. Science (New York, N.Y 306, 2087-2090 (2004).
12 Harrison, P. M. & Arosio, P. The ferritins: molecular properties, iron storage function and cellular regulation. Biochimica et biophysica acta 1275, 161-203 (1996).
13 Kontoyiannis, D., Pasparakis, M., Pizarro, T. T., Cominelli, F. & Kollias, G. Impaired on/off regulation of TNF biosynthesis in mice lacking TNF AU-rich elements: implications for joint and gut-associated immunopathologies. Immunity 10, 387-398 (1999).
14 Solomon, L. et al. The dextran sulphate sodium (DSS) model of colitis: an overview. Comparative Clinical Pathology 19, 235-239, doi:10.1007/s00580-010-0979-4 (2010).
15 Müllner, E. W., Neupert, B. & Kühn, L. C. A specific mRNA binding factor regulates the iron-dependent stability of cytoplasmic transferrin receptor mRNA. Cell 58, 373-382, doi:10.1016/0092-8674(89)90851-9 (1989).
16 Harford, J. B. & Klausner, R. D. Coordinate post-transcriptional regulation of ferritin and transferrin receptor expression: the role of regulated RNA-protein interaction. Enzyme 44, 28-41 (1990).
17 Pantopoulos, K. Iron metabolism and the IRE/IRP regulatory system: an update. Ann N Y Acad Sci 1012, 1-13, doi:10.1196/annals.1306.001 (2004).
18 Rouault, T. A. Post-transcriptional regulation of human iron metabolism by iron regulatory proteins. Blood cells, molecules & diseases 29, 309-314 (2002).
19 Mahler, G. J., Shuler, M. L. & Glahn, R. P. Characterization of Caco-2 and HT29-MTX cocultures in an in vitro digestion/cell culture model used to predict iron bioavailability. J Nutr Biochem 20, 494-502, doi:10.1016/j.jnutbio.2008.05.006 (2009).
20 Leonard, F., Collnot, E. M. & Lehr, C. M. A three-dimensional coculture of enterocytes, monocytes and dendritic cells to model inflamed intestinal mucosa in vitro. Molecular pharmaceutics 7, 2103-2119, doi:10.1021/mp1000795 (2010).
21 Antunes, F., Andrade, F., Araujo, F., Ferreira, D. & Sarmento, B. Establishment of a triple co-culture in vitro cell models to study intestinal absorption of peptide drugs. European journal of pharmaceutics and biopharmaceutics: official journal of Arbeitsgemeinschaft fur Pharmazeutische Verfahrenstechnik e.V 83, 427-435, doi:10.1016/j.ejpb.2012.10.003 (2013).
22 Tanoue, T., Nishitani, Y., Kanazawa, K., Hashimoto, T. & Mizuno, M. In vitro model to estimate gut inflammation using co-cultured Caco-2 and RAW264.7 cells. Biochem Biophys Res Commun 374, 565-569 (2008).
23 Lund, S. et al. The dynamics of the LPS triggered inflammatory response of murine microglia under different culture and in vivo conditions. J Neuroimmunol 180, 71-87, doi:10.1016/j.jneuroim.2006.07.007 (2006).
24 Hobbs, S., Reynoso, M., Geddis, A. V., Mitrophanov, A. Y. & Matheny, R. W., Jr. LPS-stimulated NF-κB p65 dynamic response marks the initiation of TNF expression and transition to IL-10 expression in RAW 264.7 macrophages. Physiol Rep 6, e13914, doi:10.14814/phy2.13914 (2018).
25 Domscheit, H., Hegeman, M. A., Carvalho, N. & Spieth, P. M. Molecular Dynamics of Lipopolysaccharide-Induced Lung Injury in Rodents. Frontiers in physiology 11, 36, doi:10.3389/fphys.2020.00036 (2020).
26 Gray, N. K. et al. Recombinant iron-regulatory factor functions as an iron-responsive-element-binding protein, a translational repressor and an aconitase. A functional assay for translational repression and direct demonstration of the iron switch. European journal of biochemistry/FEBS 218, 657-667, doi:10.1111/j.1432-1033.1993.tb18420.x (1993).
27 Bouton, C., Chauveau, M. J., Lazereg, S. & Drapier, J. C. Recycling of RNA binding iron regulatory protein 1 into an aconitase after nitric oxide removal depends on mitochondrial ATP. The Journal of biological chemistry 277, 31220-31227, doi:10.1074/jbc.M203276200 (2002).
28 Tannahill, G. M. et al. Succinate is an inflammatory signal that induces IL-10 through HIF-1α. Nature 496, 238-242, doi:10.1038/nature11986 (2013).
29 Starzynski, R. R. et al. Down-regulation of iron regulatory protein 1 activities and expression in superoxide dismutase 1 knock-out mice is not associated with alterations in iron metabolism. The Journal of biological chemistry 280, 4207-4212, doi:10.1074/jbc.M411055200 (2005).
30 Torti, F. M. & Torti, S. V. Regulation of ferritin genes and protein. Blood 99, 3505-3516 (2002).
31 Ruscitti, P. et al. Pro-inflammatory properties of H-ferritin on human macrophages, ex vivo and in vitro observations. Scientific reports 10, 12232, doi:10.1038/s41598-020-69031-w (2020).
32 Canal, F., Fosset, C., Chauveau, M. J., Drapier, J. C. & Bouton, C. Regulation of the cysteine desulfurase Nfs1 and the scaffold protein IscU in macrophages stimulated with interferon-gamma and lipopolysaccharide. Arch Biochem Biophys 465, 282-292, doi:10.1016/j.abb.2007.06.003 (2007).
33 Giatromanolaki, A. et al. Hypoxia inducible factor 1alpha and 2alpha overexpression in inflammatory bowel disease. J Clin Pathol 56, 209-213, doi:10.1136/jcp.56.3.209 (2003).
34 Mastrogiannaki, M., Matak, P. & Peyssonnaux, C. The gut in iron homeostasis: role of HIF-2 under normal and pathological conditions. Blood 122, 885-892, doi:10.1182/blood-2012-11-427765 (2013).
35 Olson, T. S. et al. The primary defect in experimental ileitis originates from a nonhematopoietic source. The Journal of experimental medicine 203, 541-552, doi:10.1084/jem.20050407 (2006).
36 Roulis, M., Armaka, M., Manoloukos, M., Apostolaki, M. & Kollias, G. Intestinal epithelial cells as producers but not targets of chronic TNF suffice to cause murine Crohn-like pathology. Proceedings of the National Academy of Sciences of the United States of America 108, 5396-5401, doi:10.1073/pnas.1007811108 (2011).
37 Choi, E. Y. et al. Iron chelator triggers inflammatory signals in human intestinal epithelial cells: involvement of p38 and extracellular signal-regulated kinase signaling pathways. J Immunol 172, 7069-7077 (2004).
38 Ghosh, M. C. et al. Tempol-mediated activation of latent iron regulatory protein activity prevents symptoms of neurodegenerative disease in IRP2 knockout mice. Proceedings of the National Academy of Sciences of the United States of America 105, 12028-12033, doi:10.1073/pnas.0805361105 (2008).
39 Medzhitov, R. The spectrum of inflammatory responses. Science (New York, N.Y 374, 1070-1075, doi:10.1126/science.abi5200 (2021).
40 Werner, T. et al. Intestinal epithelial cell proteome from wild-type and TNFDeltaARE/WT mice: effect of iron on the development of chronic ileitis. Journal of proteome research 8, 3252-3264, doi:10.1021/pr800772b (2009).
41 Bessman, N. J. et al. Dendritic cell-derived hepcidin sequesters iron from the microbiota to promote mucosal healing. Science (New York, N.Y 368, 186-189, doi:10.1126/science.aau6481 (2020).
42 Das, N. K. et al. Microbial Metabolite Signaling Is Required for Systemic Iron Homeostasis. Cell metabolism 31, 115-130.e116, doi:10.1016/j.cmet.2019.10.005 (2020).
43 Sanchez, K. K. et al. Cooperative Metabolic Adaptations in the Host Can Favor Asymptomatic Infection and Select for Attenuated Virulence in an Enteric Pathogen. Cell 175, 146-158.e115, doi:10.1016/j.cell.2018.07.016 (2018).
44 Kuno, S., Fujita, H., Tanaka, Y. K., Ogra, Y. & Iwai, K. Iron-induced NCOA4 condensation regulates ferritin fate and iron homeostasis. EMBO reports, e54278, doi:10.15252/embr.202154278 (2022).

1. (canceled)

2. An in vitro method of assaying inflammation in culture, the method comprising:
(a) contacting a test agent with a model for inflammation comprising tissue cells and immune cells, wherein said tissue cells and immune cells are of different species and are in direct contact; and
(b) analysing an inflammation parameter in said tissue cells and/or immune cells.

3. The method of claim 2, wherein said inflammation parameter is associated with iron homeostasis.

4-5. (canceled)

6. The method of claim 3, wherein said inflammation parameter associated with iron homeostasis is an iron-regulated gene.

7. The method of claim 6, wherein said iron-regulated gene is selected from the group consisting of ferritin, Transferrin Receptor (TfR)1, NCOA4, IRP1 and IRP2.

8. The method of claim 2, wherein said analyzing is following at least 4 hours from the contacting.

9. The method of claim 2, wherein said analyzing expression of said iron-regulated gene is at the protein level.

10. The method of claim 2, wherein said analyzing expression of said iron-regulated gene is at the mRNA level.

11. The method of claim 2, wherein said iron-regulated gene is ferritin and said analysing is by analysing ferritin-iron complex in said immune cells and wherein inflammation is indicated by an increase in said ferritin-iron complex in said immune cells, wherein said increase is with respect to control non-inflamed cells of the same type.

12. The method of claim 7, wherein said iron-regulated protein is TfR1, wherein inflammation is indicated by a decrease in said TfR1 expression in the immune cells and wherein said decrease is with respect to control non-inflamed cells of the same type or wherein said iron-regulated protein is IRP2 and wherein inflammation is indicated by a decrease in said IRP2 expression in the immune cells, wherein decrease is with respect to control non-inflamed cells of the same type.

13. (canceled)

14. The method of claim 7, wherein said iron-related protein is ferritin and analyzing is by analyzing H-subunit (FTH1) and L-subunit (FTL) thereof, wherein inflammation is indicated wherein FTH1:FTL protein ratio above 1 preferably above 2 in the immune cells.

15. The method of claim 2, wherein said inflammation parameter associated with iron homeostasis is a decrease in cellular or mitochondrial aconitase activity of IRP1 in the immune cells and the tissue cells, wherein said decrease is with respect to control non-inflamed cells of the same type.

16. The method of claim 2, wherein said inflammation parameter associated with iron homeostasis is a protein involved in Fe—S cluster assembly and wherein inflammation is indicated when a decrease in the immune cells is indicated, wherein a decrease is with respect to control non-inflamed cells of the same type.

17. The method of claim 16, wherein said gene is Nfs1 or IscU.

18. The method of claim 2, wherein said tissue cells are GI cells.

19. The method of claim 2, wherein said tissue cells are epithelial cells or fibroblasts.

20. (canceled)

21. The method of claim 19, wherein said epithelial cells have a decreased iron amount with respect to non-inflamed epithelial cells of the same type, and optionally wherein said tissue I a GI tissue, an eye tissue and a brain tissue.

22. The method of claim 2, wherein said tissue cells are selected from the group consisting of hepatocytes, neurons, cartilage, osteoblasts and fibroblasts.

23. The method of claim 22, wherein tissue cells have a decreased iron amount with respect to non-inflamed epithelial cells of the same type, and optionally wherein said tissue comprises an epithelial barrier.

24. (canceled)

25. The method of claim 3, wherein said inflammation parameter is a pro-inflammatory cytokine and/or an anti-inflammatory cytokine, wherein an increase in said pro-inflammatory cytokine and/or a decrease in anti-inflammatory cytokine in said immune cells and/or tissue cells is indicative of inflammation, wherein said increase or decrease is with respect to control non-inflamed cells of the same type.

26. The method of claim 3, wherein said inflammation parameter is a signalling pathway associated with inflammation, optionally wherein said signalling is a MAP-kinase pathway, wherein an increase in signalling via said MAP-kinase pathway n said immune cells and/or tissue cells is indicative of inflammation, wherein said increase is with respect to control non-inflamed cells of the same type.

27-29. (canceled)


SEQUENCE LISTING (58 sequences)

SEQ ID NO: 1 - DNA, 20 bp, synthetic construct
  source 1..20: mol_type = other DNA
ccagtgttaccaccaagaag

SEQ ID NO: 2 - DNA, 20 bp, synthetic construct
  source 1..20: mol_type = other DNA
acccaagaacaagcacaagg

SEQ ID NO: 3 - DNA, 21 bp, synthetic construct
  source 1..21: mol_type = other DNA
caaattcgagtgacaagcctg

SEQ ID NO: 4 - DNA, 18 bp, synthetic construct
  source 1..18: mol_type = other DNA
gagatccatgccgttggc

SEQ ID NO: 5 - DNA, 20 bp, synthetic construct
  source 1..20: mol_type = other DNA
caatttctcatgccgcacag

SEQ ID NO: 6 - DNA, 21 bp, synthetic construct
  source 1..21: mol_type = other DNA
agctggaagatcgaaagtccg

SEQ ID NO: 7 - DNA, 20 bp, synthetic construct
  source 1..20: mol_type = other DNA
gcccatcctctgtgactcat

SEQ ID NO: 8 - DNA, 20 bp, synthetic construct
  source 1..20: mol_type = other DNA
aggccacaggtattttgtcg

SEQ ID NO: 9 - DNA, 22 bp, synthetic construct
  source 1..22: mol_type = other DNA
agccagagtccttcagagagat

SEQ ID NO: 10 - DNA, 24 bp, synthetic construct
  source 1..24: mol_type = other DNA
tgttaggagagcattgagaattgg

SEQ ID NO: 11 - DNA, 21 bp, synthetic construct
  source 1..21: mol_type = other DNA
gtggagtatcacttcctgtcg

SEQ ID NO: 12 - DNA, 23 bp, synthetic construct
  source 1..23: mol_type = other DNA
ccccagaagatatgtcggaaagg

SEQ ID NO: 13 - DNA, 22 bp, synthetic construct
  source 1..22: mol_type = other DNA
catcctggtcctgatcgtctgc

SEQ ID NO: 14 - DNA, 25 bp, synthetic construct
  source 1..25: mol_type = other DNA
ccacatagagtgccacatgccctag

SEQ ID NO: 15 - DNA, 23 bp, synthetic construct
  source 1..23: mol_type = other DNA
aggctttggcttccaacttcagc

SEQ ID NO: 16 - DNA, 25 bp, synthetic construct
  source 1..25: mol_type = other DNA
aagcacaacagccttatgccgaaag

SEQ ID NO: 17 - DNA, 20 bp, synthetic construct
  source 1..20: mol_type = other DNA
aagtgcgccagaactaccac

SEQ ID NO: 18 - DNA, 20 bp, synthetic construct
  source 1..20: mol_type = other DNA
ttcagagccacatcatctcg

SEQ ID NO: 19 - DNA, 20 bp, synthetic construct
  source 1..20: mol_type = other DNA
aatcaggccctcttggatct

SEQ ID NO: 20 - DNA, 20 bp, synthetic construct
  source 1..20: mol_type = other DNA
ggttgcccatcttcttgatg

SEQ ID NO: 21 - DNA, 20 bp, synthetic construct
  source 1..20: mol_type = other DNA
aagaaagtgggaagcctcag

SEQ ID NO: 22 - DNA, 20 bp, synthetic construct
  source 1..20: mol_type = other DNA
agatcacaaactgctgggag

SEQ ID NO: 23 - DNA, 20 bp, synthetic construct
  source 1..20: mol_type = other DNA
tacatgtcacagtcaacgcc

SEQ ID NO: 24 - DNA, 20 bp, synthetic construct
  source 1..20: mol_type = other DNA
ctcgtctagagagcttgggt

SEQ ID NO: 25 - DNA, 20 bp, synthetic construct
  source 1..20: mol_type = other DNA
aatgaaagactcgcagacgc

SEQ ID NO: 26 - DNA, 21 bp, synthetic construct
  source 1..21: mol_type = other DNA
aagtcaccttgttcaaaagcg

SEQ ID NO: 27 - DNA, 20 bp, synthetic construct
  source 1..20: mol_type = other DNA
gattgtggacgccagattca

SEQ ID NO: 28 - DNA, 19 bp, synthetic construct
  source 1..19: mol_type = other DNA
ggcttcctccaccgttttc

SEQ ID NO: 29 - DNA, 20 bp, synthetic construct
  source 1..20: mol_type = other DNA
ataatccgcagacaggtcca

SEQ ID NO: 30 - DNA, 20 bp, synthetic construct
  source 1..20: mol_type = other DNA
ttccttgtgctggcttccta

SEQ ID NO: 31 - DNA, 22 bp, synthetic construct
  source 1..22: mol_type = other DNA
ggtgagaaggactggcagaaat

SEQ ID NO: 32 - DNA, 23 bp, synthetic construct
  source 1..23: mol_type = other DNA
cttgtcgatgaagtacagggcta

SEQ ID NO: 33 - DNA, 24 bp, synthetic construct
  source 1..24: mol_type = other DNA
atgagttccaagctggccgtggct

SEQ ID NO: 34 - DNA, 24 bp, synthetic construct
  source 1..24: mol_type = other DNA
tctcagccctcttcaaaaacttct

SEQ ID NO: 35 - DNA, 20 bp, synthetic construct
  source 1..20: mol_type = other DNA
ctgccattctgatttgctgc

SEQ ID NO: 36 - DNA, 20 bp, synthetic construct
  source 1..20: mol_type = other DNA
cgtacagttctagagagagg

SEQ ID NO: 37 - DNA, 20 bp, synthetic construct
  source 1..20: mol_type = other DNA
ttgggcatagagaccccgtt

SEQ ID NO: 38 - DNA, 23 bp, synthetic construct
  source 1..23: mol_type = other DNA
gcacattgctcagttcatacacc

SEQ ID NO: 39 - DNA, 23 bp, synthetic construct
  source 1..23: mol_type = other DNA
tggagactttggatcggttggtg

SEQ ID NO: 40 - DNA, 21 bp, synthetic construct
  source 1..21: mol_type = other DNA
cagtggctggcagaaaccttg

SEQ ID NO: 41 - DNA, 25 bp, synthetic construct
  source 1..25: mol_type = other DNA
caccatgacaggaacctattctggc

SEQ ID NO: 42 - DNA, 22 bp, synthetic construct
  source 1..22: mol_type = other DNA
aaccactcgggcaaagcgtgac

SEQ ID NO: 43 - DNA, 26 bp, synthetic construct
  source 1..26: mol_type = other DNA
aaacgaagtcaaccaaggctacagtc

SEQ ID NO: 44 - DNA, 26 bp, synthetic construct
  source 1..26: mol_type = other DNA
acaggagtgcaaggaactggagatag

SEQ ID NO: 45 - DNA, 27 bp, synthetic construct
  source 1..27: mol_type = other DNA
aaacgcgaattcaccatgacgaccgcg

SEQ ID NO: 46 - DNA, 27 bp, synthetic construct
  source 1..27: mol_type = other DNA
accgcggccgcttagctttcattatca

SEQ ID NO: 47 - DNA, 20 bp, synthetic construct
  source 1..20: mol_type = other DNA
gaaccaggcccttttggatc

SEQ ID NO: 48 - DNA, 21 bp, synthetic construct
  source 1..21: mol_type = other DNA
ccaggaagtcacagagatggg

SEQ ID NO: 49 - DNA, 22 bp, synthetic construct
  source 1..22: mol_type = other DNA
caaattcctgagcacttgatgg

SEQ ID NO: 50 - DNA, 21 bp, synthetic construct
  source 1..21: mol_type = other DNA
aaagggacagctacaataccg

SEQ ID NO: 51 - DNA, 20 bp, synthetic construct
  source 1..20: mol_type = other DNA
actagcagaggaaacgctgg

SEQ ID NO: 52 - DNA, 20 bp, synthetic construct
  source 1..20: mol_type = other DNA
ggaataggccaaggaagaca

SEQ ID NO: 53 - DNA, 21 bp, synthetic construct
  source 1..21: mol_type = other DNA
actcccggacacatgcttatg

SEQ ID NO: 54 - DNA, 19 bp, synthetic construct
  source 1..19: mol_type = other DNA
gctgacgagcacgttccat

SEQ ID NO: 55 - DNA, 19 bp, synthetic construct
  source 1..19: mol_type = other DNA
ggcgctgactgcgtgttaa

SEQ ID NO: 56 - DNA, 23 bp, synthetic construct
  source 1..23: mol_type = other DNA
aggtcaccttgctccattatttg

SEQ ID NO: 57 - DNA, 21 bp, synthetic construct
  source 1..21: mol_type = other DNA
aaagccaagagagaccttgga

SEQ ID NO: 58 - DNA, 20 bp, synthetic construct
  source 1..20: mol_type = other DNA
ctaggtcctgggcatgtctc